SEQUENCE LISTING (24 sequences)

SEQ ID NO: 1 - protein, 586 aa, Saccharomyces cerevisiae
MSRSNSIYTEDIEMYPTHNEQHLTREYTKPDGQTKSEKLNFEGAYINSHGTLSKTTTREIEGDLDSETSSHSSDDKVDPTQQITAETKAPYTLLSYGQKWGMVAILTMCGFWSSLGSPIYYPALRQLEKQFNVDENMVNVTVVVYLLFQGISPTVSGGLADCFGRRPIILAGMLIYVIASIGLACAPSYGVIIFLRCIQSIGISPTIAISSGVVGDFTLKHERGTFVGATSGFVLLGQCFGSLIGAVLTARWDWRAIFWFLTIGCGSCFLIAFLILPETKRTIAGNLSIKPKRFINRAPIFLLGPVRRRFKYDNPDYETLDPTIPKLDLSSAGKILVLPEIILSLFPSGLLFAMWTLMLSSISSGLSVAPYNYHLVIIGVCYLPGGIGGLMGSFFTGRIIDMYFKRKIKKFEQDKANGLIPQDAEINMFKVRLVCLLPQNFLAVVAYLLFGWSIDKGWRIESILITSFVCSYCAMSTLSTSTTLLVDLYPTKSSTASSCFNFVRCSLSTIFMGCFAKMKAAMTVGGTFTFLCALVFFFNFLMFIPMKYGMKWREDRLLKQQRQSWLNTLAVKAKKGTKRDQNDNHN

SEQ ID NO: 2 - DNA, 1761 bp, Saccharomyces cerevisiae
atgtcacgaagtaacagtatatacacagaagatattgagatgtatcctactcacaatgagcagcatctgacgagagaatacacaaaaccagatggtcaaacgaaaagtgaaaaactaaatttcgaaggcgcttatatcaacagtcacgggactctatccaagactaccacaagggaaatagaaggcgatttagactctgagacttcttctcactccagtgatgataaagttgatccaacccaacagataactgcagagacaaaagcgccatatactttactaagttacggtcagaagtggggaatggttgcaattctaacaatgtgtggcttttggtcttcattgggatcaccgatctattatcccgctttgaggcagttggaaaagcaatttaatgtggatgaaaatatggttaacgttactgtggttgtgtatctgttgtttcaaggtatatctcccacagttagcggtggtctggcggattgctttggacggagacctataattttagcaggtatgctgatatacgttattgcatccatcgggctagcatgtgctccatcatacggtgttattatcttcttgaggtgtattcagagtattggtatttcccccacaattgcaatcagttctggtgttgtcggggacttcactttgaagcatgaaagaggtacatttgttggcgccacctcagggtttgttttactgggtcaatgttttggtagtcttattggtgccgtcctgactgcaaggtgggattggagagctatcttttggtttttgaccattggttgtggaagttgttttctaattgcctttcttattttgccagaaacaaagcgaaccattgcaggtaacctttccattaagcctaaaagattcatcaacagagcgccgattttcctactgggcccagttagaaggagattcaagtacgataatcctgattatgagacattagatcccaccattccgaaactagatttgtcatccgccgggaagatccttgtattgccagaaattattctgtctttatttccatcagggctcttatttgctatgtggacgttaatgctttcctctatttcatccggtttatcagtggcgccatacaactatcatttggtcattattggtgtgtgttatctacccggtggtattggcggtttaatgggttccttcttcacaggtagaattattgacatgtatttcaagagaaagatcaagaaattcgagcaagacaaagctaacgggttgattccacaagacgcagagattaatatgttcaaagtccgtttagtttgtcttctgccccaaaatttcttggccgtcgtagcctatcttttatttggttggagtatagataagggctggaggatcgaatctattttaattacttcatttgtctgttcatactgtgctatgagtacattatcaacatcaactacgttattggtcgatttgtacccaacgaaatcatctacagcaagtagttgtttcaactttgttaggtgcagtttgagtaccatcttcatgggttgctttgccaaaatgaaagctgcgatgactgtaggtggtacttttacatttttatgtgcgctggtatttttcttcaacttcttaatgtttattccgatgaaatatggtatgaagtggagggaggatagattattgaaacaacaaagacagtcttggttaaacaccttggcagtcaaagccaaaaagggaacaaaaagagaccagaacgataatcataattaa

SEQ ID NO: 3 - protein, 563 aa, Saccharomyces cerevisiae
MTKQQTSVMRNASIAKEEREGSDNNNVDRSSSDAISDNDAERSNSHSEIDNESNFDMVPYSRFSHKQKMLLVVQCAFTGFFSTVAGSIYYPVLTIIERKFNITEELANVTIVVYFIFQGVAPSIMGGLADTFGRRPIVLWAILAYFCACIGLACAHNYAQILALRCLQAAGISPVIAINSGIMGDVTTKVERGGYVGLVAGFQVVGTAFGALIGAGLSSKWGWRAIFWFLAIGSGICLVFSTLLMPETKRTLVGNGSVTPRSFLNRSLILHVGSVKKTLHLDDPDPETLEPRTSVDFLAPLKILHIREIDILLSIAGLQFSTWTTHQTALTIVLSKKYNLSVAKIGLCFLPAGISTLTSIISAGRYLNWSYRTRKVKYNRWIKEQELQLMEKYKGDKNKVAELIHSNSHYAFNLVEARLHPAFVTLLLSSIGFTAFGWCISVKTPLAAVLCTSAFASLFSNCILTFSTTLIVDLFPSKASTATGCLNLFRCLLSAIFIAALTKMVEKMRYGGVFTFLSAITSSSSLLLFYLLKNGKQLSFDRIRANDKSAGRSVGKNSEKVST

SEQ ID NO: 4 - DNA, 1692 bp, Saccharomyces cerevisiae
atgaccaaacaacaaacttctgtaatgcgtaacgcatctatagccaaggaagaacgtgaaggatctgataataataatgtggacagatccagctcagatgccatcagtgacaacgacgcagaaagatctaattctcattctgaaatagataacgaatcaaattttgatatggtgccttattcaaggttcagccataagcaaaaaatgcttcttgtcgttcagtgtgccttcacaggctttttttcaaccgtggcagggtctatctactatccagtgcttactattattgagaggaaatttaacatcacagaagagttagctaatgtcactattgtggtttattttattttccaaggggtggctccttcaattatgggtggtttagctgatacatttggaaggcggcctattgtattatgggcaatcttagcctacttctgtgcctgcatcggactggcatgtgcacataattacgcccaaatcctggctttaaggtgtttacaggcagcaggtatttctcctgttattgcaataaatagcggaataatgggagatgtaacgacaaaagtagagaggggtggatatgttggattggttgcagggttccaagttgttggaactgcattcggtgcattgattggtgccggattatcctcgaaatggggttggagggcaattttttggtttttggctattggttccggaatttgtctagttttttcaaccttattgatgccagagacaaaaagaactttggtgggaaacggctccgtaacaccaaggtcatttttgaataggtctttgatattgcatgttggttccgtaaaaaaaacattacatttagacgatcctgaccctgagaccttagaaccacgtactagcgtggattttttagcacctttaaaaattttgcacatacgagaaatagacattttattgtccattgccggcctgcaattttccacgtggaccactcatcaaacagccttaacaattgtattgagtaaaaagtacaatctatctgttgcaaaaataggcctgtgctttttacctgcaggaatctcgacactgacaagtattatctctgctggtcggtacttaaattggagctacagaacaagaaaggttaaatacaacagatggattaaagaacaagaattacagctgatggagaaatataaaggtgataaaaacaaggtagcagaacttattcactctaattctcattatgcattcaatttagtggaggcaaggttgcatccagcatttgtgacacttcttctcagtagcattggctttactgcctttggatggtgtatttcggtgaaaacaccgcttgctgctgtattatgcacaagtgcatttgcaagtttattttcaaactgtattttgaccttctctactactttgatagtagatctctttccgtctaaggcttctaccgcaacaggttgcttaaatttatttagatgtctcctatccgcaattttcattgcagcactgaccaagatggtcgagaagatgagatatggaggtgtttttacgtttttgagtgctataacttcatcttcatcgcttttattattctatctcctgaagaatgggaaacaactatcgttcgataggattagggcaaatgataagtcggctggaaggtcagttggtaaaaattcagaaaaagtttcgacgtga

SEQ ID NO: 5 - protein, 542 aa, Saccharomyces cerevisiae
MAGATSSIIRENDFEDELAESMQSYNRETADKLALTRTESVKPEPEITAPPHSRFSRSFKTVLIAQCAFTGFFSTIAGAIYYPVLSVIERKFDIDEELVNVTVVVYFVFQGLAPTFMGGFADSLGRRPVVLVAIVIYFGACIGLACAQTYAQIIVLRCLQAAGISPVIAINSGIMGDVTTRAERGGYVGYVAGFQVLGSAFGALIGAGLSSRWGWRAIFWFLAIGSGICFLASFLILPETKRNISGNGSVTPKSYLNRAPILVLPTVRKSLHLDNPDYETLELPTQLNLLAPFKILKAYEICILMLVAGLQFAMYTTHLTALSTALSKQYHLTVAKVGLCYLPSGICTLCSIVIAGRYLNWNYRRRLKYYQNWLGKKRSKLLEEHDNDLNLVQRIIENDPKYTFNIFKARLQPAFVTLLLSSSGFCAYGWCITVKAPLAAVLCMSGFASLFSNCILTFSTTLIVDLFPTKTSTATGCLNLFRCILSAVFIAALSKMVEKMKFGGVFTFLGALTSSSSILLFILLRKGKELAFKRKKQELGVN

SEQ ID NO: 6 - DNA, 1629 bp, Saccharomyces cerevisiae
atggcaggagcaacatcaagtataattcgggaaaatgattttgaggacgagcttgcggagagtatgcagtcatataatagagaaactgcagacaaattggctttgactaggaccgagagtgtaaagccagaaccggagataaccgctccgcctcactcacgcttttcccgttctttcaagacagttttaatagctcagtgcgctttcactgggtttttctccacaatagcaggtgccatctactatccagttctgagcgttatagaaagaaaattcgatattgacgaggaattggtgaatgtcactgttgtagtatattttgtatttcagggtcttgcccccacattcatgggcgggtttgccgattcactgggcaggagaccggtggtgcttgtcgcaatcgtcatttattttggtgcctgcatcggtcttgcttgtgctcaaacgtatgctcagatcattgtgctaagatgtctacaagccgccggtatttcacccgtgattgcgattaacagcggaataatgggagatgttactactagagccgagcgcggcgggtacgttggatatgttgctggatttcaagttctaggttctgcgttcggagcccttatcggtgccggattatcatctagatggggatggagagccatcttttggttcttagcaattggatctggcatttgcttcctagcctcgtttttaattttgccagaaacaaagaggaacatatccgggaatggttctgtcaccccaaaatcatacttaaatagagctcccattcttgttttgccaacagtaagaaaatcattgcatttggataatccagattacgaaacattggaactgcccacgcaactaaatctactggcgcccttcaagattttgaaagcttatgaaatttgtatacttatgctagtcgctggattacaatttgccatgtatactacgcaccttacggcgttatccacagctttgagtaaacaatatcacttgactgttgcaaaggtaggattatgctaccttccctccggtatctgcacattatgtagtattgtcattgctggaagatatttgaattggaattacaggcgcagattaaaatattaccaaaattggttgggcaagaagagatcaaagcttttagaagaacacgacaacgaccttaatttggtgcaacgtatcatagaaaatgatcccaaatacacctttaatatcttcaaggcgaggttgcaacccgcatttgttaccttgcttttaagcagtagcggattttgtgcgtacggatggtgcattactgtcaaagcccctttagcggccgttctttgcatgagtggatttgcgtcgctgttttccaattgcattttgactttttcaacaactctaatagttgatctttttcccacgaagacatctacggctacgggatgtttaaaccttttcagatgcattctatctgctgtgtttatagctgcattgagtaaaatggtagaaaaaatgaaattcggaggtgtttttacatttttgggggctttaacctcctcgtcatcgatcctcttatttattctcttgagaaaaggtaaagaattagcctttaagaggaaaaagcaagaactgggagtaaattaa

SEQ ID NO: 7 - protein, 689 aa, Saccharomyces cerevisiae
MQAQGSQSNVGSLRSNCSDNSLPNNHVMMHCDESSGSPHSEHNDYSYEKTNLESTASNSREHRDNQLSRLKSEEYVVPKNQRRGLLPQLAIIPEFKDARDYPPMMKKMIVFLIAFSSMMGPMGTSIIFPAINSITTEFKTSVIMVNVSIGVYLLSLGVFPLWWSSLSELEGRRTTYITSFALLFAFNIGSALAPDINSFIALRMLCGAASASVQSVGAGTVADLYISEDRGKNLSYYYLGPLLAPLLSPIFGSLLVNRWPWRSTQWFMVILSGCNVILLTVLLPETLRKQDSKGAIAQILAERRIQVDNNERGEIQEDYQRGEDETDRIENQVATLSTEKHNYVGEVRDQDSLDLESHSSPNTYDGRAGETQLQRIYTEASRSLYEYQLDDSGIDATTAQVTRIRSTDPKLARSIRENSLRKLQTNLEEQVKKVLSSNGGEIAPKQVSAVRKVWDTFFVYFIKPLKSLHFLEYPPVALAITFSAISFSTVYFVNMTVEYKYSRPPYNFKPLYIGLLYIPNSVTYFFASIYGGRWVDMLLKRYKEKYGILAPEARISWNVVTSVISFPIALLIFGWCLDKKCHWVTPLIGTALFGYAAMMTIGATLSYLVDSLPGKGATGVALNNLIRQILAATAVFVTTPMLNGMGTGWAFTMLAFIVLGASSVLIILKKHGDYWRENYDLQKLYDKID

SEQ ID NO: 8 - DNA, 2070 bp, Saccharomyces cerevisiae
atgcaagcccaaggttcacaatcgaatgtagggtctttgaggagtaattgctctgacaattcactaccgaacaatcatgttatgatgcactgcgatgaaagcagcggcagcccgcacagcgagcacaacgattatagttacgaaaagaccaatctggaaagtacggcatcaaatagtcgtgaacacagagacaaccagctaagtaggttgaagagtgaggaatacgttgttccaaagaatcaacgtaggggactattgcctcaactcgccattataccggagttcaaggatgccagagattatccaccgatgatgaaaaagatgattgtcttcttgattgcgttttcctccatgatgggccccatgggcacatctatcatttttccagcgatcaactcaatcacaacagaatttaaaacatcagtgattatggtaaacgtttcaattggtgtgtaccttttaagtcttggtgttttcccattgtggtggtcttctctatccgagctagagggcagaagaactacttacataacttcatttgcattattgtttgcatttaatatcgggtctgctctagctcctgatatcaactcatttattgccttgagaatgctctgtggggctgcttctgccagtgttcaaagtgtaggtgctggaacagtggctgatttatatattagcgaagatagaggtaaaaatttgagttattactatttgggtccactactggcgccgctactatctccaatttttggatctttgttagtgaatcgctggccctggagatccactcaatggtttatggttattttatccggatgtaatgtcattcttttgacggtgttactacctgaaacattaagaaaacaagattctaaaggcgctatcgctcaaattttggctgaaagacgtattcaagtagacaataacgaacgtggagagatacaagaagattatcagaggggagaagatgagacagatcgaattgaaaaccaagttgccacattatctactgagaagcataactacgttggagaggtaagggatcaagactcgctagatttagaaagtcactctagccccaatacttatgatggtcgagctggagaaacccaattgcaacggatttatacagaggcgagtagaagtctgtatgaatatcagctagatgatagcggtatcgatgcaacaacagcacaagttacgagaataagatcaacagatccaaagttagcgagatcgattcgagaaaatagtctgagaaaattacaaaccaacctggaagagcaagtcaaaaaagtgctatccagtaatggaggtgaaatcgctcctaaacaggtatcagcggtgaggaaggtctgggacaccttttttgtttattttatcaagcctttaaaatcattgcacttcctagaatatccacccgtggcacttgcaataacattttccgcaatttccttttccacagtatactttgttaatatgacagttgaatataaatattcaaggcctccttacaactttaaaccattatacattggtctactgtatattccgaattctgtaacatactttttcgcctcaatttacggtggacgttgggtggacatgcttttaaaaagatacaaagagaaatatggaattcttgctcctgaagctcgtatatcgtggaatgttgttacatctgtaatatctttccccattgcgctattgatatttggctggtgcctagataaaaaatgccactgggtaacgccactaattggaacagccctctttggatatgcagctatgatgacaattggtgctaccctttcctatttagtcgattcattgccgggaaagggtgccaccggtgttgctttgaataatttaataaggcaaatcttggctgcaaccgcagtctttgtcaccacacccatgttaaacggtatgggaactgggtgggctttcacaatgctggcctttatcgtcttgggtgctagcagtgtacttataatactgaaaaagcacggtgattactggagagagaactacgatttacaaaaattgtacgacaaaattgattaa

SEQ ID NO: 9 - protein, 586 aa, Saccharomyces cerevisiae
MSDHSPISNKENHLLPSDSSRSSSSDMHSTGTTGTTGVEPVDFTGEGAKYTTATEGNGGADLAIQRTTTMNSAAESEVNITRRLTKILTGSVNEPDRVEVDYTNCAPMGGDRPYPPSLPSRDLYEVTFDGPNDPLHPFNWPMKKKVLLCLVLCLDSIAIAMCSSIFASAVPQICEIYHVIEVVAILGITLFVLGFAASPVIYAPLSELYGRKGVLVLSAFGFALFQFAVATAENLQTIFICRFFGGFIGAAPMAVVPAAFADMFDTNVRGKAIALFSLGVFVGPILSPVMGSYIAQRTTWRWLEYVVGCFASAVFVAIVLFFEETHHPTILVNKAKQMRKQSNNWGIHAAHEDVELSIKDIVQKTVTRPIIMLFVEPLLLFVTIYNSFVYGILYLLLEAYPLVFVEGYGFTENGELPYIALIIGMMVCAAFIWYMDNDYLKRCRAKGKLVPEARLYAMVIAGTVFPIGILWFCWTGYYPHKIHWMVPTVGGAFIGFGLMGIFLPCLNYIIESYLLLAASAVAANTFMRSAFGACFPLFAGYMFRGMGIGWAGLLLGLFAAAMIPVPLLFLKYGESIRKKSKYAYAA

SEQ ID NO: 10 - DNA, 1761 bp, Saccharomyces cerevisiae
atgtcggatcattctcccatttctaataaggaaaatcatctgctgccatcagactcgtcaagaagctcatccagcgatatgcattcgacgggcaccacgggaaccacaggcgtcgaacctgtagatttcaccggtgaaggtgccaaatacacaactgctacggagggcaatggtggtgcagatttagcgattcaaagaacgacgactatgaattctgctgcagaatcagaggttaatatcacgagaagattaactaaaatccttactgggtctgttaacgagcctgaccgtgtagaggttgattataccaattgtgcgcccatgggtggtgacagaccttaccctccatcgttgccgagcagagacctgtacgaggttacttttgatggtcctaacgacccactacatccatttaactggcccatgaagaagaaagtgctgctatgtctggtcttatgtctggattctattgccattgctatgtgttcttccatttttgcctctgcagtgccgcaaatctgcgagatataccacgtcatcgaagttgtcgccattttgggtatcacgctttttgttcttgggtttgcggcctcaccggttatctatgctcctctttctgaattgtacggtagaaagggtgttctggttttatctgcgtttggatttgcccttttccaatttgctgtcgctactgctgagaacctgcaaactattttcatatgtagattctttggtggttttatcggggcagcacccatggccgtcgtccccgccgcgtttgccgacatgtttgatactaatgttagaggtaaagccattgcgctattttctctaggtgtttttgtaggccccattttatcgcctgtcatgggttcttatatcgcacaaagaactacctggagatggttagaatatgttgtcggttgtttcgcttccgcagttttcgttgccatcgtattgttctttgaagaaacacatcaccctaccattttggttaacaaggccaaacagatgagaaagcaaagtaataactggggtattcatgctgctcatgaagacgtggagctatccatcaaagacattgtccaaaaaactgtgacgaggcctatcattatgcttttcgtggaaccattgctactattcgtgactatttacaactcttttgtctacggtatcttgtatttgttactggaagcctacccacttgtctttgtggagggttatgggtttactgaaaacggtgagttgccatacatcgccttgattatcggtatgatggtgtgtgctgctttcatttggtatatggacaacgattatttgaaaagatgtagagccaaggggaaattagtgcccgaggccagattgtacgcaatggtcattgcaggtaccgttttccctattggtatcttatggttctgttggacgggctactatcctcacaagattcattggatggtccccacagtaggaggggccttcatcgggttcggtttaatgggtattttcttgccatgtttaaactatatcattgaatcgtatctattgttggcagcttctgccgtcgcagcaaacactttcatgaggtctgcatttggtgcatgcttcccattgtttgcaggatatatgttccgtggcatgggtatcggttgggctggtttgttattaggtctatttgccgctgcgatgattcccgtgcctttactattcttaaaatatggtgaatctatcagaaagaaatccaagtatgcttacgccgcttaa

SEQ ID NO: 11 - protein, 614 aa, Saccharomyces cerevisiae
MSDQESVVSFNSQNTSMVDVEGQQPQQYVPSKTNSRANQLKLTKTETVKSLQDLGVTSAAPVPDINAPQTAKNNIFPEEYTMETPSGLVPVATLQSMGRTASALSRTRTKQLNRTATNSSSTGKEEMEEEETEEREDQSGENELDPEIEFVTFVTGDPENPHNWPSWVRWSYTVLLSILVICVAYGSACISGGLGTVEKKYHVGMEAAILSCSLMVIGFSLGPLIWSPVSDLYGRRVAYFVSMGLYVIFNIPCALAPNLGCLLACRFLCGVWSSSGLCLVGGSIADMFPSETRGKAIAFFAFAPYVGPVVGPLVNGFISVSTGRMDLIFWVNMAFAGVMWIISSAIPETYAPVILKRKAARLRKETGNPKIMTEQEAQGVSMSEMMRACLLRPLYFAVTEPVLVATCFYVCLIYSLLYAFFFAFPVIFGELYGYKDNLVGLMFIPIVIGALWALATTFYCENKYLQIVKQRKPTPEDRLLGAKIGAPFAAIALWILGATAYKHIIWVGPASAGLAFGFGMVLIYYSLNNYIIDCYVQYASSALATKVFLRSAGGAAFPLFTIQMYHKLNLHWGSWLLAFISTAMIALPFAFSYWGKGLRHKLSKKDYSIDSVEM

SEQ ID NO: 12 - DNA, 1845 bp, Saccharomyces cerevisiae
atgagtgatcaagaatctgttgtttcattcaactcacaaaacacttccatggtggacgttgagggccaacaacctcaacagtatgtcccctcaaaaaccaactctcgtgcaaatcaacttaagttaactaagactgagaccgtcaagtctttacaagatttaggtgtcacttcagccgccccagtgcctgatatcaatgcgccacaaactgctaagaataacattttccctgaagaatataccatggaaacaccatctgggctggttccagtggctaccttacaatctatgggtagaaccgcctctgccttatctcgtactagaacaaagcaattgaaccgtaccgctaccaattcctcatccacaggtaaagaagaaatggaagaggaagaaactgaagaacgtgaagaccagagcggtgaaaacgagctagatccagagatcgaattcgttacttttgttactggtgatccagaaaaccctcacaattggccctcatgggttcgttggagttacactgtcctgttgtccatcttagttatttgcgttgcctacggttctgcttgtatcagtggtgggttgggaaccgttgaaaagaaataccatgtaggtatggaagccgctattttatcatgttctttaatggttattgggttctcgctgggtcctttgatttggtctcctgttagtgatctttacggtagaagagttgcttactttgtttctatgggtctttatgtcatcttcaatatcccttgcgccttagctccaaatctaggttgtcttttagcttgtagatttttatgtggtgtttggtcatcatctggtttgtgtttagttggtgggtctattgccgatatgttcccaagtgaaacaagaggtaaggctattgctttcttcgcttttgctccttacgttggtcccgttgttggtccactagttaacggttttatttccgtttctaccggacgtatggacctgattttctgggtcaatatggcctttgcaggtgttatgtggatcatatcttctgccatcccagaaacgtacgctccagttatcttgaagagaaaggctgctagattaagaaaggaaactggtaatcccaagattatgactgagcaggaagcgcaaggtgtcagtatgagtgaaatgatgagggcttgtctgttgagacctttgtacttcgctgtcactgaacctgttctagttgccacttgtttctacgtgtgtttgatttactctctactatatgcgttcttctttgccttccctgtcattttcggtgaactatatggctacaaagataaccttgtgggtttaatgtttattcctattgttatcggtgctctttgggcgttagccacaactttctactgtgaaaacaaatatttacaaattgtcaaacagcgtaaacctactcctgaagatcgtttgctaggtgctaagatcggtgctccatttgctgcaattgctctatggatcctgggtgctaccgcttataaacatattatttgggttggtccagcttcagctggtttagcttttggtttcggtatggtgttgatttattattcattgaataattacattattgattgctacgtccaatacgcatccagtgctctggctacaaaggttttcttaagatccgccggtggtgctgccttccccttgtttaccattcaaatgtaccacaaattgaatttgcactggggttcttggttgttggctttcatctccactgctatgattgctttaccttttgcattttcttactggggtaagggcttgagacataagttgtccaagaaggactattccatcgacagtgttgagatgtaa

SEQ ID NO: 13 - protein, 622 aa, Saccharomyces cerevisiae
MNRQESINSFNSDETSSLSDVESQQPQQYIPSESGSKSNMAPNQLKLTRTETVKSLQDMGVSSKAPVPDVNAPQSSKNKIFPEEYTLETPTGLVPVATLHSIGRTSTAISRTRTRQIDGASSPSSNEDALESDNNEKGKEGDSSGANDEAPDLDPEIEFVTFVTGDPENPHNWPAWIRWSYTVLLSILVICVAYGSACISGGLGTVEKKYHVGMEAAILSVSLMVIGFSLGPLIWSPVSDLYGRRVAYFVSMGLYVIFNIPCALAPNLGSLLACRFLCGVWSSSGLCLVGGSIADMFPSETRGKAIAFFAFAPYVGPVVGPLVNGFISVSTGRMDLIFWVNMAFAGVMWIISSAIPETYAPVILKRKAARLRKETGNPKIMTEQEAQGVSMGEMMRACLLRPLYFSVTEPVLVATCFYVCLIYSLLYAFFFAFPVIFGELYGYKDNLVGLMFIPIVIGALWALATTFYCENKYLQIVKQRKPTPEDRLLGAKIGAPFAAIALWILGATAYKHIIWVGPASAGLAFGFGMVLIYYSLNNYIIDCYVQYASSALATKVFLRSAGGAAFPLFTIQMYHKLNLHWGSWLLAFISTAMIALPFAFSYWGKGLRHKLSKKDYSIDSIE

SEQ ID NO: 14 - DNA, 1869 bp, Saccharomyces cerevisiae
atgaacagacaggaatccataaattcgtttaattcagacgaaacatcttcgttgtctgatgtagaaagtcagcagccgcaacaatatatcccttcagagagtggatctaaatccaacatggctcctaatcaactgaagttgacccggacggaaaccgtgaagtcattgcaggacatgggtgtgagctccaaagcccccgttcctgatgttaatgctcctcaatctagcaagaataagatttttcctgaagaatatactttagaaacccctacaggtttagttcctgtcgccactctacattccataggtagaacttctactgcgatttcccgtacgagaactagacagatcgatggcgcttcttcgccttcttctaatgaagatgctttagaaagtgataataacgaaaagggtaaagaaggcgactctagtggtgcgaatgacgaagctccagatctagatccggaaattgaattcgttacctttgtgactggcgatccagaaaacccccataactggcctgcatggatccgttggagttacactgtcctactgtcaatcttagttatttgtgtcgcctacgggtctgcttgtatcagtggtgggttgggaaccgttgaaaagaaataccatgtaggtatggaagccgctattttatcggtttctttaatggttattgggttctcgctgggtcctttgatttggtctcctgttagtgatctttacggtagaagagttgcttactttgtttctatgggtctttatgtcatcttcaatatcccttgcgccttagctccaaatctaggtagtcttttagcttgtagatttttatgtggtgtttggtcatcatctggtttgtgtttagttggtgggtctattgccgatatgttcccaagtgaaacaagaggtaaggctattgctttcttcgcttttgctccttacgttggtcccgttgttggtccactagttaacggttttatttccgtttctaccggacgtatggacctgattttctgggtcaatatggcctttgcaggtgttatgtggatcatatcttctgccatcccagaaacgtacgctccagttatcttgaagagaaaggctgctagattaagaaaggaaactggtaatcccaagattatgactgagcaggaagcgcaaggtgtcagtatgggtgaaatgatgagggcttgtctgttgagacctttgtacttctctgtcactgaacctgttctagttgctacttgtttctacgtgtgtttgatttactctctactatatgcgttcttctttgccttccctgtcattttcggtgaactatatggctacaaagacaaccttgtgggtttgatgtttattcctattgttatcggtgctctttgggcgttagccacaactttctactgtgaaaacaagtatttacaaattgtcaaacagcgtaaacctactcctgaagatcgtttgctaggtgctaagatcggtgctccatttgctgcaattgctttatggatcctgggtgctaccgcttataaacatattatttgggttggtccagcttcagctggtttagcttttggtttcggtatggtgttgatttattattcattgaataattacattattgattgctacgtccaatacgcatccagtgctctggctacaaaggttttcttaagatccgccggtggtgctgccttccccttgtttaccattcaaatgtaccacaaattgaatctgcactggggttcttggttgttggctttcatctccactgctatgattgctttaccttttgcattttcttactggggcaaaggtttgagacataagttatccaagaaggattattccatagatagtattgaataa

SEQ ID NO: 15 - protein, 659 aa, Saccharomyces cerevisiae
MPSSLTKTESNSDPRTNIQQVPKALDKNVTNSGNLDSTSSSTGSITEDEKRSEPNADSNNMTGGEPIDPRDLDWDGPDDPDNPHNWSSLKKWYTTMTSAFLCLVVTMGSSLYVSSVPELVERYHVSQTLALAGLTFYLLGLSTVIGAPLSEVFGRKPVYLFSLPVSMLFTMGVGLSNGHMRIILPLRFLSGVFASPALSVGSGTILDIFDVDQVSVAMTYFVLSPFLGPVLSPIMAGFATEAKGWRWSEWIQLIAGGLILPFIALMPETHKGIILRKRAKKRNIALKKFSREAQKEFLKTTVTITILRPLKMLVVEPIVFVFSVYVAFIFAILFGFFEAYAVIYRGVYHMSMGISGLPFIGIGVGLWIGAFFYLYIDRKYLFPKPPAGTQPLTEKERTSKRTTPYRGARDAETGELLPVVPEKFLIACKFGSVALPIGLFWQAWTARSDVHWMAPVAAGVPFGFGLILIFFSVLMYFSTCYPPLTVASCLAANNLLRYVMSSVFPLFTIQMYTKMKIKWASTLFALVCVVMIPIPWVFEKWGSKLRHKSQFGYAAMEKEAETEGGIDDVNAVDGELNLTRMTTLRTMETDPSTREKPGERLSLRRTHTQPVPASFDREDGQHAQNRNEPISNSLYSAIKDNEDGYSYTEMATDASARMV

SEQ ID NO: 16 - DNA, 1980 bp, Saccharomyces cerevisiae
atgccttcttctttgacaaaaaccgaatctaactcagatcctcgaacaaacatacagcaggtaccaaaagccttggacaaaaatgtaactaatagcggcaacttggattcgacttcttcttcgacagggtcaataacagaggacgaaaaaagatcagaaccgaatgcagattccaataacatgacagggggcgagccaattgacccaagagatctcgattgggacgggcccgatgacccagacaatccgcataattggtcctctttgaagaaatggtatactactatgacttcagcttttctttgtttggtcgtcacaatgggtagttccctatatgtttcttccgtaccagaattggtagaaagatatcacgttagtcagactttggcgcttgctggtttaactttctatcttctgggtttatcgactgtcatcggcgctccattaagtgaagtgtttggtcgtaagcctgtctatttattttccctacctgtgtcaatgcttttcaccatgggtgttgggttatccaatggccacatgagaataattctacccctaaggtttttatcaggtgtctttgcatcccccgccttatccgttggatcaggtaccatcctggatatctttgacgtggaccaggtctccgtcgcaatgacatactttgttctatctccattcttagggccagttttatctcctattatggcaggatttgctactgaagccaaaggctggcgttggtctgaatggatccaattgattgcgggcggtctaatcttgccatttattgcgctcatgccagagacccataaaggtattattttaagaaagcgtgccaagaagagaaatatagccttgaagaagttcagcagggaagctcagaaagaatttttaaaaaccactgtcactattacaatcttaaggcctttaaaaatgcttgtcgttgaacctattgtatttgttttcagtgtttatgtcgctttcatattcgccattttatttggatttttcgaagcatacgctgtcatttatcgtggtgtttaccacatgtctatggggatatctggcttgccatttattggtattggtgtcgggttatggataggtgctttcttttacctttacatcgacagaaaatatctcttccctaaaccaccagctggcactcaaccactcaccgaaaaagagagaacttctaaaaggactaccccttacagaggtgcaagagacgctgaaacaggtgaattgctacccgttgttccagaaaaatttttaattgcctgtaaatttggttctgtagctttgcctatcggcctgttttggcaagcctggactgctagatcagatgtccactggatggctcctgtggctgccggtgtgccatttggttttggtttaattttgatttttttcagcgttttaatgtacttctctacatgctatccgccattaactgtggcatcctgtttggctgccaataatctattaagatacgttatgagtagtgtattcccattgtttaccatccaaatgtacacaaagatgaaaatcaaatgggcaagcactttatttgcattagtttgcgttgtgatgattccaattccatgggtttttgaaaaatgggggagcaaattaagacacaagtcacaatttggttatgccgccatggaaaaggaggctgagaccgaaggtggtatagatgacgtaaatgctgtcgatggtgaattgaatctaacaaggatgaccacgttaaggaccatggaaacagacccttcgactagagaaaaaccaggtgaaaggctatctctgcgcaggacccatacgcagcccgttcctgcctcgtttgatcgcgaggacgggcaacatgcgcaaaatcgtaatgaacccatttccaatagtttatactcagctatcaaggataatgaagacggttattcgtatacggaaatggccaccgatgcttccgccagaatggtttga

SEQ ID NO: 17 - protein, 548 aa, Saccharomyces cerevisiae
MVYTSTYRHTIVVDLLEYLGIVSNLETLQSAREDETRKPENTDKKECKPDYDIECGPNRSCSESSTDSDSSGSQIEKNDPFRVDWNGPSDPENPQNWPLLKKSLVVFQIMLLTCVTYMGSSIYTPGQEYIQEEFHVGHVVATLNLSLYVLGYGLGPIIFSPLSETARYGRLNLYMVTLFFFMIFQVGCATVHNIGGLIVMRFISGILCSPSLATGGGTVADIISPEMVPLVLGMWSAGAVAAPVLAPLLGAAMVDAKNWRFIFWLLMWLSAATFILLAFFFPETQHHNILYRRALKLRKETGDDRYYTEQDKLDREVDARTFLINTLYRPLKMIIKEPAILAFDLYIAVAYGCFYLFFEAFPIVFVGIYHFSLVEVGLAYMGFCVGCVLAYGLFGILNMRIIVPRFRNGTFTPEAFLIVAMCVCWCLPLSLFLFGWTARVHWILPVISEVFFVLAVFNIFQATFAYLATCYPKYVASVFAGNGFCRASFACAFPLFGRAMYDNLATKNYPVAWGSSLVGFLTLGLAIIPFILYKYGPSLRTRSSYTEE

SEQ ID NO: 18 - DNA, 1647 bp, Saccharomyces cerevisiae
atggtatacacttcaacgtacagacacactatcgttgttgaccttttagaatatttgggtatagtgtccaacttagaaactttacagagtgcccgtgaagatgaaacaagaaaacccgagaataccgataaaaaagaatgtaaacccgactatgatatagaatgcggtcctaatagatcgtgctctgaatcctctaccgattcagactctagtggttcacagatcgaaaaaaatgatcctttcagggtggattggaacggccccagtgatcctgagaacccacaaaactggcccctactgaaaaaatcattggtagtattccaaataatgttacttacttgcgtcacgtacatgggatcctccatttacacacctggccaggaatatattcaagaagagttccacgttggtcatgtagtggcaacattaaatctttctttatatgttcttggttatggtctaggtcccatcattttttcaccgctatcagaaactgcacgctatggccgtctaaatctgtacatggtgactttattttttttcatgatctttcaagttggttgtgctactgtgcataacatcggcggtttaatcgtcatgcgtttcatcagtggcatactgtgcagcccatcgttggccactggtggcggtacagtggctgatatcatttcaccagaaatggttcctctcgttttaggtatgtggtcagccggtgctgttgctgcgccagtcttggctcccttactaggcgctgctatggtcgatgctaaaaattggcgattcatattttggttattaatgtggttaagtgctgccacttttatcttgttggcatttttcttccctgaaacacaacaccataatattttgtaccgccgtgctttgaaattgagaaaagaaactggtgatgacaggtactatactgaacaggataaactcgatagagaagttgatgcaagaacttttttgatcaatactttgtataggcctctcaaaatgattatcaaagagcctgcaattttggcttttgatctctatatcgctgttgcttatggttgtttctacttattctttgaagcattccctattgtatttgtaggtatataccacttcagcttagttgaagttggcttggcctatatggggttttgcgtagggtgcgtacttgcttatggcttattcggtattttaaacatgaggattattgtaccacgttttagaaacggcacattcaccccggaagcttttttaatcgtggcaatgtgtgtctgctggtgcctgcctctgtctttgttcttatttggttggactgctcgagtgcattggattttgccagttatctcggaagttttttttgttttagctgtctttaacattttccaagcaacttttgcatatttggctacatgctacccaaagtatgttgcatccgtttttgcaggcaatggtttttgtcgggcttcgtttgcctgtgcttttccgttgtttggtagagcaatgtatgacaatttagctactaagaactatcctgtggcatggggttcgtccttagtggggttcctaactttaggtctagctattatcccgtttatactttataagtatgggccatcattacgtacaagatcttcgtacacagaggagtag

SEQ ID NO: 19 - protein, 514 aa, Saccharomyces cerevisiae
MVAEFQIASAQSSALTSTEEEHCSINSDKAAKLDLELTSERKNDGKQSHEVTFNEDIADPEDIARHMSTARRYYISSLITFTSMVITMISSSWTLPSTHIIEHFHISHEVSTLGITLYVFGLGIGPLFLSPLSELYGRRITFLYALTLSIIWQCLTIWSKTITGVMFGRFLSGFFGSAFLSVAGGAIADIFDKDQIGIPMAIYTTSAFLGPSLGPIIGGALYHQSYKWTFITLLITSGCCLVMIIFTIPETYKPMLLIRKAKRLRKEKNDQRYYAVLEVTREQTSLLSAIFLSTKRPFGLLLRDRMMGVLCFYTGLELAIIYLYFVAFPYVFKKLYNFGPMEIACSYIGIMVGMILSAPTCLLFQKTFEWRVKRNNGVKTPEMRFEPLFYGAFLTPVGLFIFAFTCYKHVHWIAPIIGSAIFGSGVYFVFTGVFAYTVDAYRRYAASGMACNTFVRCIMAGVFPLFGLQMYKSMGVNWAGFLLAMVTVAMIPVPFLFTKYGARLRAKSPYAWDD

SEQ ID NO: 20 - DNA, 1545 bp, Saccharomyces cerevisiae
atggttgcagaatttcaaatagcgagtgcacagtcatcggcattgacatctactgaagaagagcattgttcaataaactcagacaaagccgcaaaactggatctggaattgaccagtgaacgaaaaaatgacggcaaacaatcgcatgaagtgaccttcaacgaggacatcgcggacccagaagatatagcacggcatatgagcaccgcccgccggtattatatttcctcgttaattacattcacatcgatggtaattacgatgatctcgtccagctggacgcttccttcaacgcacataatcgaacactttcatatttcgcacgaagtcagcactctgggaattacgctttatgtgtttgggctcggtataggacctttatttctgtctccactaagtgaactatatggacgaaggatcacatttttatacgcccttacgctcagtatcatatggcagtgtttgaccatttggtccaagaccattacgggtgtcatgtttggcagatttctatccgggttttttggttcagcctttctaagtgtggctggcggcgccattgccgatatattcgacaaagaccaaattggtattcctatggcaatatacactacatcggcctttttggggccctctttaggaccaatcattggtggagccttataccatcaaagttataaatggacatttatcacacttctcatcacttctggatgttgtctcgttatgatcatctttaccatacctgaaacctacaaaccaatgctgttaatacgtaaagctaagagattgaggaaagagaaaaatgatcaacggtattatgctgtcctggaagtcacccgcgaacagacttccttactttccgcaatctttctctcaaccaaaagaccctttggcctgttacttcgagaccgaatgatgggtgtgctttgcttctatactggattggaacttgccataatatatttatatttcgtagcgtttccttatgtgttcaagaaactttacaactttgggcccatggagattgcatgctcctatatcggtattatggtcggcatgatactctcggctcctacttgcttgttattccaaaaaacgtttgagtggagagtcaaaagaaataatggcgtcaagactcccgaaatgagattcgagcctctgttctacggcgcctttttgaccccagttgggctcttcatttttgcattcacctgctacaagcatgtccattggattgcaccaattataggcagtgcaattttcggttcaggtgtttatttcgtctttaccggtgtttttgcgtatacagtggatgcctatagaagatatgcagcttctggaatggcttgtaacacatttgtgagatgcataatggccggtgtcttccctcttttcggactccagatgtacaagtcaatgggagtgaattgggccggctttctcttggcaatggtgaccgtagcgatgattcctgtcccatttctgtttaccaaatacggtgcccgactgagagccaagtcgccgtatgcatgggatgattga

SEQ ID NO: 21 - protein, 572 aa, Saccharomyces cerevisiae
MGSEPFQKKNLGLQINSQESGTTRSTFHSLEDLGDDVINESWDQVNQKRANIDHDVFHEHPDSSPSLSAQKAKTKEEEVAVKSSNSQSRDPSPDTQAHIPYTYFSKDQRLIIFGIIIFIGFLGPMSGNIYIPALPLLQREYDVSATTINATVSVFMAVFSVGPLFWGALADFGGRKFLYMVSLSLMLIVNILLAAVPVNIAALFVLRIFQAFASSSVISLGAGTVTDVVPPKHRGKAIAYFMMGPNMGPIIAPIVAGLILMKGNYWRWLFGFTSIMTGIALILVTALLPETLRCIVGNGDPKWGDKKDERENNESPFFEGNKISHRRLFPDIGIRKPVNNDAFFQENFPKPPKAGLTLYWKMIKCPPIIITSVSTALLFSSYYAFSVTFSYYLEHDYRFTMLEIGAAYVCPGVAMLLGSQSGGHLSDYLRSRWIKSHPKKKFPAEFRLLLNLIGILLTICGTIGYGWAIFFHYHFVVLLVFSALTAFGMTWCSNTSMTYLTELFPKRAAGTVAVSSFFRNVGAAISSAIILQLCNAMGIGWCFTGLGLCSSISLIGILYLLIFQRKYTAKEF

SEQ ID NO: 22 - DNA, 1719 bp, Saccharomyces cerevisiae
atgggaagcgaaccgtttcagaaaaagaatttgggtctgcaaattaattcgcaagaaagtggaacaacccgctcaacatttcattcgctagaagatctaggagatgatgtaattaatgaaagctgggatcaggtgaaccaaaagagagccaatatagatcatgatgtctttcatgagcaccctgactcttccccatcattgtcagcacagaaagcaaaaacaaaagaagaggaagttgctgtaaagtcatcgaactcccagtcaagagacccttctcctgatactcaagcacatattccatatacttatttttccaaggatcaaagactaatcatttttggaattatcatttttataggatttttgggcccaatgtctggaaacatatatataccggctttaccattgctgcaaagggaatatgatgtaagtgcaacaacaataaacgctacagtttctgtatttatggctgttttttccgttggtccattgttttggggcgcactggcggattttggtggaaggaaattcttatatatggtgtcgttatcactaatgttaattgttaatatacttttggccgctgtaccagtcaatattgctgccctttttgttttaagaattttccaagctttcgcttccagctctgtgatttctctgggagctggcactgtaacagacgttgttcctccaaaacacaggggaaaggccatagcgtatttcatgatgggtccaaacatgggtcctattatagcacccattgttgctgggcttattttaatgaaaggaaattactggagatggctttttggtttcacttctatcatgacaggaatagcattgatcttggttactgctttacttccagaaacgctacgttgtatagttggtaatggagaccctaaatggggtgacaaaaaagatgaacgtgaaaataacgaatctccattcttcgaaggtaataaaatatcacatcggcgtctgttcccagacattggtatccgtaaaccagtcaataatgatgctttcttccaagaaaattttccaaagccgcctaaagcaggtttgacactatattggaaaatgattaaatgtcctccaataataatcacgtccgtcagtactgcactcctgttctccagttactatgcgttcagcgtcacgttttcgtattaccttgaacatgactaccgttttactatgttagaaattggtgctgcttatgtctgcccaggtgtagctatgttactaggatctcaatctggtgggcacctctcagattatcttcgttcacgctggatcaaaagtcatcctaaaaagaaattcccggcagagtttcgtttattactgaacctaattggaattttactaacaatatgtggcacaataggatacggatgggcaatcttctttcattatcattttgtggttcttttagttttttccgctctcactgcctttggtatgacctggtgcagcaacacatcaatgacatatttaactgagttattccccaaaagagctgctggaactgttgctgttagtagtttctttcgaaatgtgggcgctgccattagttccgctatcattttacagctctgtaacgcaatgggaattggatggtgttttacagggctcggtctctgcagttcaatttcattgattggtatattatatcttctcatttttcaaagaaaatatactgccaaagaattttaa

SEQ ID NO: 23 - protein, 586 aa, Saccharomyces cerevisiae
MDKYTNRDHPDYIPGTFNIYSSQNLENGIIYESKLKKTSSGVVLIPQPSYSPNDPLNWSSWRKLAHFGLMAFITAFTAATSNDAGAAQDSLNEIYGISYDSMNTGAGVLFLGIGWSTLFLAPFANLYGRKITYIVCTTLGLFGALWFALAKRTSDTIWSQLFVGISESCAEAQVQLSLSDIFFQHQLGSVLTVYIMCTSIGTFLGPLIAGYISAFTNFRWVGWVAVIISGGLLITIIFGCEETYFDRGQYMTPLTSCQSGYEDGTTLQNSDNTAVSRRKRHLDAKLSTPGAMGEKGVDLSETAEFEVNNEEEVTIPETRELIDGSKEHLKPYPKRVAILTKATNLKGYGFKQYFKYLKINLRMFLFPPVWLSGMFWGIQDVFLTFYLTTQESAYYEPPWNYSDFGVAIMNVPTLIGAVIGCICAGIVSDYFVLWMARHNRGILEAEFRLYFSIATAIIGPAGLLMFGIGTARQWPWQAIYVGLGFVGFAWGCSGDIAMAYLMDCYPDMVLEGMVCTAIINNTISCIFTFTCSDWLAASGTENTYIALAVINFGITAFALPMYYYGKRIRLWTKRWYLQSVNLRDGV

SEQ ID NO: 24 - DNA, 1761 bp, Saccharomyces cerevisiae
atggacaaatataccaacagggatcatccggactatattcctggcacatttaacatctattcttcccaaaatttggaaaatggtattatatacgaatcaaaattgaagaaaacctcctcaggagtcgtcctaatccctcaaccatcatactcgccaaatgatccactgaactggtctagctggaggaaattggctcattttggtctaatggcctttataactgcgtttaccgccgctactagtaacgacgctggtgctgctcaagactcattgaatgagatttatgggatatcctacgattccatgaacacaggggctggtgttcttttcctaggtattggttggtccactttattcttggctccatttgctaacttgtatggcaggaagattacgtacatagtttgcaccacattaggtctctttggtgcgctttggtttgctttggccaaaagaacgagcgacacaatatggtctcagttgtttgtaggtattagtgagtcctgtgctgaagcccaggtgcaactgtccctgagcgacatttttttccagcaccaattgggctccgtattgaccgtgtatatcatgtgcactagtatcggtacgttcttgggcccattgatcgctgggtacatatctgcatttaccaacttccgttgggtcggttgggtcgcagtgatcatatctggtggccttttaataactattatatttggctgcgaagaaacgtacttcgacagaggccagtatatgacccctttgaccagctgtcaatcgggatacgaagacggtaccactttacaaaactctgacaatacggccgtgtcgcgcaggaaacgtcatcttgacgctaaattatcaactcctggagccatgggtgagaaaggtgtagacctttcagagacggctgaattcgaagttaacaatgaagaggaagttaccatacctgagactcgcgaattgattgatggttcaaaagagcatttgaaaccatacccaaaaagagtagcaatattaaccaaagctactaatttgaaaggctacggttttaaacagtatttcaaatatctaaagatcaaccttagaatgttcttattcccgccagtgtggttgtctggtatgttttggggtattcaagacgttttcctgacgttttatttgaccactcaagaaagcgcctactacgaacctccatggaactatagtgattttggtgtcgcaattatgaatgttcccacacttattggagcagtcatcggttgtatctgtgctggcattgttagtgactactttgttctttggatggctcgtcacaatagaggaattttagaggcagaatttagactatacttctctatcgcaactgcaattattgggccagcgggtttgctgatgtttggtatcggtaccgctagacaatggccttggcaagctatatacgtcgggttgggttttgttgggtttgcatggggttgttctggtgatattgcgatggcgtatttaatggattgttaccccgatatggttttggaaggtatggtttgtactgctattattaacaacacgatatcttgcattttcacctttacctgttctgattggctagctgcatctggtactgaaaatacttacattgctttagctgtcatcaactttgggattactgcatttgctttaccaatgtactactatggtaagaggataagactttggactaagagatggtatttgcaatctgtcaatttgagagacggtgtgtaa

(19) United States
(12) Patent Application Publication
(10) Pub. No.: US 2023/0183302 A1
(43) Pub. Date: Jun. 15, 2023

(54) Yeast strains for reducing contamination by lactic acid bacteria

(71) Applicant: Université Libre de Bruxelles, Bruxelles (BE)

(72) Inventors: Bruno André, Landelies (BE); Christos Gournas, Attica (GR)

(73) Assignee: Université Libre de Bruxelles, Bruxelles (BE)

(21) Appl. No.: 17/921,372

(22) PCT Filed: Apr. 26, 2021
(86) PCT No.: PCT/EP2021/060851

§ 371 Date: Oct. 26, 2022

(30) Foreign Application Priority Data
Apr. 27, 2020 (EP) 20386020.0

(51) Int. Cl. C07K 14/395; C12P 7/06; C12N 1/18; C12P 7/16; C12P 7/04

(52) U.S. Cl. CPC C07K 14/395; C12P 7/06; C12N 1/18; C12P 7/16; C12P 7/04

(57) ABSTRACT

Described herein is a yeast strain wherein activity of one or more membrane transporters of the DHA1 family is reduced relative to a wild type strain or to a parental strain from which it is derived.

### FIELD

Aspects and embodiments described herein relate to the field of industrial microbiology and biotechnology.

### BACKGROUND

Yeasts such as Saccharomyces cerevisiae are used in many industrial production processes, for instance bio-ethanol production. Bacterial contamination is a frequently encountered problem in yeast fermentation tanks. Lactic acid bacteria (LAB), especially Lactobacillus sp., are the primary contaminants of concern in industrial ethanol fermentation processes (Beckner et al., 2011, Skinner and Leathers, 2004). LAB contamination reduces yeast growth and ethanol yield (Narendranath et al., 1997). Any solution applicable on an industrial scale to prevent or limit the spread of these bacteria would represent a high added value. Previously proposed solutions include the use of antibiotics. However, antibiotics are costly and their utilization in industrial processes is in question. Antiseptics have also been used to limit the spread of LAB but these substances can also affect yeast growth. Moreover, calibrating their optimal concentration is not easy. Therefore, there is still a need for better methods to control LAB infection in yeast fermentation processes.

### SUMMARY

Most lactic acid bacteria (LAB) are auxotrophic for several amino acids. In other words, they are incapable of producing them and must therefore take them out of the external environment to multiply. Pioneering works established that yeast growing under suboptimal conditions excrete amino acids and other substances able to crossfeed Lactobacillus cultivated in the same defined medium (Challinor and Rose, 1954). These observations have been confirmed in several subsequent studies. In particular, a recent work based on metabolomics, transcriptomics and genetic analysis showed that S. cerevisiae, in a nitrogen-rich environment, adjusts its metabolism by secreting several metabolites, especially amino acids, thereby promoting growth in the same medium of Lactobacillus sp. (Ponomarova et al., 2017).

Excretion of amino acids by yeast cells was reported to be favored by their overproduction, impairment of their uptake, and/or situations of metabolic imbalance (Grenson, 1973). However, the mechanisms underlying this excretion have long remained unknown. In 2004, the inventors identified and characterized AQR1, the first yeast gene encoding a membrane transport protein that promotes excretion of certain amino acids (Velasco et al., 2004). This protein belongs to the DHA1 (“Drug:H+-Antiporter-1”) family of membrane transporters, which includes multiple members.

The inventors have now surprisingly found a solution for eliminating, limiting or preventing the propagation of LAB in large-scale yeast cultures by employing yeast strains with reduced amino acid excretion, wherein activity of one or more membrane transporters of the DHA1 family is reduced.

The aspects and embodiments as described herein are associated with at least the following advantages:
effectively limit LAB contamination in yeast fermentation processes without the need of antibiotics or antiseptics
increasing production yield, such as ethanol yield

Accordingly, the aspects and embodiments of the present invention as described herein solve at least some of the problems and needs as discussed herein.

An aspect of the invention relates to a yeast strain wherein activity of one or more membrane transporters of the DHA1 family is reduced relative to a wild type strain or to a parental strain from which it is derived. In some embodiments, a yeast strain of the invention is such that one or more nucleotide sequences encoding the one or more membrane transporters of the DHA1 family are deleted or inactivated, or wherein expression of said one or more nucleotide sequences is downregulated, preferably inducibly downregulated. In some embodiments, a yeast strain of the invention is such that the one or more membrane transporters of the DHA1 family are selected from the group consisting of AQR1, QDR1, QDR2, QDR3, TPO1, TPO2, TPO3, TPO4, FLR1, YHK8, DTR1, HOL1 and/or homologues thereof. In some embodiments, a yeast strain of the invention is such that the one or more membrane transporters of the DHA1 family are encoded by a nucleotide sequence comprising, consisting essentially of or consisting of a sequence having at least 60% sequence identity with any of the nucleotide sequences set forth in SEQ ID NOs: 2, 4, 6, 8, 10, 12, 14, 16, 18, 20, 22 and 24; or comprise, consist essentially of or consist of an amino acid sequence having at least 60% identity or similarity with any of the amino acid sequences set forth in SEQ ID NOs: 1, 3, 5, 7, 9, 11, 13, 15, 17, 19, 21 and 23. In some embodiments, a yeast strain of the invention has reduced activity of at least two, at least three, at least four, at least five, at least six, or at least seven transporters of the DHA1 family relative to a wild type strain or to a parental strain from which it is derived. In some embodiments, a yeast strain of the invention is such that the one or more membrane transporters comprise AQR1, QDR2, QDR1 and TPO4 or homologues thereof, or wherein the one or more membrane transporters comprise AQR1, QDR3, QDR2, QDR1, DTR1, HOL1 and TPO1, or homologues thereof. In some embodiments, a yeast strain of the invention is a Saccharomyces species, preferably Saccharomyces cerevisiae.

Another aspect of the invention relates to a composition comprising, consisting essentially of, or consisting of a yeast strain according to the invention; and optionally one or more formulation excipients, preferably wherein the composition is a dry yeast composition or an instant yeast composition.

Another aspect of the invention relates to a method for the production of yeast cells, comprising culturing a yeast strain according to the invention in a suitable culture medium.

Another aspect of the invention relates to a method for producing a fermentation product, comprising: culturing a yeast strain according to the invention on a fermentable substrate; and, optionally, recovering the fermentation product. In some embodiments, a method for producing a fermentation product according to the invention is such that the fermentation substrate comprises, consists essentially of or consists of an unrefined or low-processed source of sugars. In some embodiments, a method for producing a fermentation product according to the invention is such that the fermentation product is an alcohol, preferably a lower alkyl alcohol such as ethanol, propanol or butanol, more preferably ethanol.

Another aspect of the invention relates to a method for the production of a product of interest, comprising:
a) optionally, transforming a yeast cell as described herein with a vector encoding the product of interest or encoding one or more enzymes capable of producing the product of interest;
b) culturing a yeast cell as described herein in a culture medium to produce the product of interest; and
c) optionally, isolating the product of interest from the yeast cell or culture medium.

Another aspect of the invention relates to a use of a yeast strain according to the invention for eliminating, reducing or preventing contamination of lactic acid bacteria in yeast cultures. In some embodiments, a use of a yeast strain according to the invention is such that the lactic acid bacteria comprise a Lactobacillus species.

### DESCRIPTION

A. Yeast Strains or Cells

In a first aspect there is provided a yeast strain, wherein activity of one or more membrane transporters of the DHA1 family is reduced relative to a wild type strain or to a parental strain from which it is derived. In some embodiments, activity is reduced relative to a control or reference strain. Throughout this disclosure, a yeast strain may be replaced by a yeast cell. Also, throughout this disclosure, a yeast strain may be understood to be an isolated yeast strain, and a yeast cell may be understood to be an isolated yeast cell.

The terms “wild-type” and “parental” may be understood as referring to a given SNP, allele, gene, genotype, phenotype or strain that was either first discovered or is the most common and is considered the reference against which other forms are compared. A “control strain” and a “reference strain” may be understood as referring to a given strain that is considered the reference against which the strain according to the invention is compared. A wild type strain or parental strain and a control strain or reference strain as used herein are described further later herein.

Membrane transporters of the DHA1 family or 12-spanner drug:H⁺ antiporter DHA1 family belong to the Major Facilitator Superfamily (MFS) transporters. DHA1 family transporters are assigned TC 2.A.1.2 in the IUBMB-approved classification system for membrane transport proteins known as the Transporter Classification (TC) system.

In some embodiments, activity of one or more membrane transporters of the DHA1 family is reduced by at least 10%, 20%, 30%, 40%, 50%, 60%, 70%, 80%, 90%, 95%, 98%, 99%, 99.9% or 100% relative to the activity in a wild type strain or in a parental strain from which it is derived, or relative to a control or reference strain. In some embodiments, activity of one or more membrane transporters of the DHA1 family is reduced from 10-20%, from 20-30%, from 30-40%, from 40-50%, from 50-60%, from 60-70%, from 70-80%, from 80-90% or from 90-100% relative to the activity in a wild type strain or in a parental strain from which it is derived, or relative to a control or reference strain. In some embodiments, activity of a membrane transporters of the DHA1 family may refer to the excretion of amino acids. Accordingly, in some embodiments, the one or more membrane transporters display at least 10%, 20%, 30%, 40%, 50%, 60%, 70%, 80%, 90%, 95%, 98%, 99%, 99.9% or 100% or from 10-20%, from 20-30%, from 30-40%, from 40-50%, from 50-60%, from 60-70%, from 70-80%, from 80-90% or from 90-100% reduced excretion of amino acids. In some embodiments, activity of a membrane transporter of the DHA1 family may refer to the excretion of at least one amino acid, such as alanine, asparagine, arginine, aspartic acid, cysteine, glutamine, glutamic acid, glycine, histidine, isoleucine, leucine, lysine, methionine, phenylalanine, proline, serine, threonine, tryptophan, tyrosine or valine. In some embodiments, activity of a membrane transporter of the DHA1 family may refer to the excretion of at least 2, at least 3, at least 4, or at least 5 amino acids, preferably selected from the group consisting of alanine, asparagine, arginine, aspartic acid, cysteine, glutamine, glutamic acid, glycine, histidine, isoleucine, leucine, lysine, methionine, phenylalanine, proline, serine, threonine, tryptophan, tyrosine and valine.

In some embodiments, amino acid excretion may be assessed by any suitable means known to the skilled person including high performance liquid chromatography (HPLC), ultra performance liquid chromatography (UPLC), gas chromatography-mass spectrometry (GC-MS), liquid chromatography-mass spectrometry (LC-MS) and other techniques including crossfeeding of amino acid auxotroph microorganisms in solid or liquid media, for example as described in Grenson, 1973 or Velasco et al., 2004, both incorporated herein by reference.

In some embodiments, activity of a membrane transporter of the DHA1 family may refer to expression. Accordingly, in some embodiments, there is provided a yeast strain, wherein expression of one or more membrane transporters of the DHA1 family is reduced relative to a wild type strain or to a parental strain from which it is derived, or relative to a control or reference strain. Expression (i.e. gene expression) may be assessed using techniques such as qRT-PCR, RNA sequencing, Northern blot analysis, Western blot analysis, mass spectrometry analysis of protein-derived peptides or ELISA as described under the section entitled “general information”.

In some embodiments, expression of one or more membrane transporters of the DHA1 family is reduced by at least 10%, 20%, 30%, 40%, 50%, 60%, 70%, 80%, 90%, 95%, 98%, 99%, 99.9% or 100% relative to the expression level in a wild type strain or in a parental strain from which it is derived, or relative to a control or reference strain.

In some embodiments, activity such as excretion of amino acids or such as expression may be assessed after 1 hour, 2 hours, 4 hours, 6 hours, 8 hours, 10 hours, 12 hours, 16 hours, 20 hours, 24 hours, 36 hours or 48 hours of culturing the yeast strain. In some embodiments, activity such as excretion of amino acids or such as expression is measured during the exponential growth phase or during the stationary growth phase. In some embodiments, activity such as excretion of amino acids or such as expression may be measured during the diauxic shift. The activity such as excretion of amino acids or such as expression level may be measured in any suitable culture medium as described elsewhere herein.

In some embodiments, a yeast strain as described herein may be a yeast strain wherein one or more nucleotide sequences encoding the one ore more membrane transporters of the DHA1 family are deleted or inactivated, or wherein expression of said one or more nucleotide sequences is downregulated. In some embodiments, inactivation of the one or more nucleotide sequences may involve an at least partial deletion or including any mutation, preferably a loss of function mutation, relative to a wild type strain or to a parental strain from which it is derived, or relative to a control or reference strain. In a preferred embodiment, the one or more nucleotide sequences encoding a membrane transporter of the DHA1 family are deleted or inactivated. In a more preferred embodiment, the one or more nucleotide sequences encoding a membrane transporter of the DHA1 family are deleted. In a preferred embodiment, downregulation may be inducible downregulation and/or temporary downregulation.

In some embodiments, there is provided a yeast strain as described herein, wherein reduced activity of one or more membrane transporters of the DHA1 family, relative to a wild type strain or to a parental strain from which it is derived, or relative to a control or reference strain, is inducible and/or temporary. In some embodiments, the expression of one or more nucleotide sequences encoding a membrane transporter of the DHA1 family can be inducibly and/or temporarily downregulated. As used herein, inducible reduction of activity or inducible downregulation of expression may be understood as a controllable means to reduce activity and/or downregulate expression. Temporary reduction of activity or temporary downregulation of expression may be understood as a reduction or downregulation which occurs only in a desired time frame. In some embodiments, inducible downregulation may be downregulation or modulation at will. Inducible and/or temporary downregulation may be achieved e.g. by replacing native promoters with inducible or repressible promoters. In some embodiments, a yeast strain as described herein may comprise a vector encoding the one or more membrane transporters of the DHA1 family. In a preferred embodiment, the one or more membrane transporters of the DHA1 family are operably linked to an inducible promoter. In another preferred embodiment, the one or more membrane transporters of the DHA1 family are operably linked to a repressible promoter. In some embodiments, the one or more membrane transporters of the DHA1 family are operably linked to a promoter which is both inducible and repressible. Any suitable inducible or repressible promoter may be used, for example as described in Ronicke et al. (1997) Use of conditional promoters for expression of heterologous proteins in Saccharomyces cerevisiae. Methods Enzymol., 283, 313-322; Redden et al. The synthetic biology toolbox for tuning gene expression in yeast, FEMS Yeast Research, Volume 15, Issue 1, February 2015, Pages 1-10; Kluge, et al. Inducible promoters and functional genomic approaches for the genetic engineering of filamentous fungi. Appl Microbiol Biotechnol 102, 6357-6372 (2018); Peng et al. Controlling heterologous gene expression in yeast cell factories on different carbon substrates and across the diauxic shift: a comparison of yeast promoter activities. Microb Cell Fact. 2015; 14:91; all of which are incorporated herein by reference. Non-limiting examples include promoters from the GAL (glucose repressed) and MET (methionine repressed) genes.

Deletion, inactivation and downregulation of expression of a gene may be achieved by any suitable means as known to a skilled person. Techniques of genetically modifying microbial organisms are well known in the art, see for example “Molecular Cloning Fourth edition, 2012 Cold Spring Harbor Laboratory Press, A laboratory manual, by M. R. Green and J Sambrook” and “Methods in Yeast Genetics and Genomics, 2015 Edition: A CSHL Course Manual, Cold Spring Harbor Laboratory Press, by Maitreya Dunham, Marc Gartenberg and Grant W. Brown”, which are incorporated herein by reference.

Gene deletion may be achieved by any suitable means as known to a skilled person including for example excising a gene using a FLP-FRT or Cre-Lox cassette, homologous recombination or CRIPR-Cas9 activity or through loss or degradation of a plasmid. Other examples include a PCR-based gene deletion strategy as described in “Baudin et al., Nucl. Acids Res. 21, 3329-3330, 1993”; “Wach et al., Yeast 10, 1793-1808, 1994” and “Gardner J. M., Jaspersen S. L. (2014) Manipulating the Yeast Genome: Deletion, Mutation, and Tagging by PCR. In: Smith J., Burke D. (eds) Yeast Genetics. Methods in Molecular Biology (Methods and Protocols), vol 1205. Humana Press, New York, N.Y.”, all of which are incorporated herein by reference. Gene deletion may also be achieved by CRISPR-Cas.

Gene inactivation may be achieved by any suitable means as known to a skilled person, including gene deletion, random mutagenesis using mutagens (such as ultraviolet rays, radiation or mutagenic compounds) or using PCR (as described in “Weir M., Keeney J. B. (2014) PCR Mutagenesis and Gap Repair in Yeast. In: Smith J., Burke D. (eds) Yeast Genetics. Methods in Molecular Biology (Methods and Protocols), vol 1205. Humana Press, New York, N.Y.”, incorporated herein by reference), or site-directed mutagenesis, for example to disturb the open reading frame. Directed mutations can, without limitation, be introduced by PCR-based methods (as described in Gardner J. M., Jaspersen S. L. (2014), incorporated herein by reference).

Gene deletion and gene inactivation can also be achieved by CRISPR-Cas based methods, for example as described in “Raschmanová et al. Implementing CRISPR-Cas technologies in conventional and non-conventional yeasts: Current state and future prospects. Biotechnol Adv. 2018; 36(3):641-665” and “Cai et al. CRISPR-mediated genome editing in non-conventional yeasts for biotechnological applications. Microb Cell Fact. 2019 Apr. 2; 18(1):63” and “Mitsui et al. CRISPR system in the yeast Saccharomyces cerevisiae and its application in the bioproduction of useful chemicals. World J Microbiol Biotechnol. 2019 Jul. 6; 35(7):111”, all of which are incorporated herein by reference.

Downregulation and inducible and/or temporary downregulation of gene expression may be achieved by any suitable means as known to a skilled person including mutating and/or replacing promoters or other regulatory sequences. Gene expression downregulation including inducible and/or temporary downregulation may also be achieved via inhibiting promoter activity, activating a transcriptional repressor, repressing a repressible promoter, decreasing RNA stability, activating a post-transcriptional inhibitor (for example, expressing a ribozyme or antisense oligonucleotide) or inhibiting translation (for example, via a regulatable RNA). It is also encompassed, in some embodiments, that the activity of the one or more membrane transporters of the DHA1 family may be reduced (and inducibly and/or temporarily reduced) by failing to make a required post-translational modification, inactivating a polypeptide (for example by binding an inhibitor or via a polypeptide-specific protease), or failing to properly localize a polypeptide.

In some embodiments, the one or more membrane transporters of the DHA1 family as described herein may be selected from the group consisting of AQR1, QDR1, QDR2, QDR3, TPO1, TPO2, TPO3, TPO4, FLR1, YHK8, DTR1, HOL1 and/or (functional) homologues thereof.

In some embodiments, the one or more membrane transporters of the DHA1 family as described herein may be selected from the group consisting of AQR1, QDR1, QDR2, QDR3, TPO1, TPO4, DTR1, HOL1 and/or (functional) homologues thereof, preferably AQR1, QDR1, QDR2, QDR3 and/or (functional) homologues thereof.

In some embodiments, the one or more membrane transporters of the DHA1 family as described herein are AQR1, QDR2 and QDR3; QDR1, QDR2 and QDR3; AQR1, QDR1, QDR2 and TPO4; or AQR1, QDR1, QDR2, QDR3, DTR1, HOL1 and TPO1.

AQR1 (Systematic name: YNL065W) refers to a protein in S. cerevisiae comprising, consisting essentially of, or consisting of the amino acid sequence of SEQ ID NO: 1, or a sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity or similarity with SEQ ID NO: 1. It is encoded by a gene comprising, consisting essentially of, or consisting of the nucleotide sequence of SEQ ID NO: 2, or a sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 2.

QDR1 (Systematic name: YIL120W) refers to a protein in S. cerevisiae comprising, consisting essentially of, or consisting of the amino acid sequence of SEQ ID NO: 3, or a sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity or similarity with SEQ ID NO: 3. It is encoded by a gene comprising, consisting essentially of, or consisting of the nucleotide sequence of SEQ ID NO: 4, or a sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 4.

QDR2 (Systematic name: YIL121W) refers to a protein in S. cerevisiae comprising, consisting essentially of, or consisting of the amino acid sequence of SEQ ID NO: 5, or a sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity or similarity with SEQ ID NO: 5. It is encoded by a gene comprising, consisting essentially of, or consisting of the nucleotide sequence of SEQ ID NO: 6, or a sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 6.

QDR3 (Systematic name: YBR043C) refers to a protein in S. cerevisiae comprising, consisting essentially of, or consisting of the amino acid sequence of SEQ ID NO: 7, or a sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity or similarity with SEQ ID NO: 7. It is encoded by a gene comprising, consisting essentially of, or consisting of the nucleotide sequence of SEQ ID NO: 8, or a sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 8.

TPO1 (Systematic name: YLL028W) refers to a protein in S. cerevisiae comprising, consisting essentially of, or consisting of the amino acid sequence of SEQ ID NO: 9, or a sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity or similarity with SEQ ID NO: 9. It is encoded by a gene comprising, consisting essentially of, or consisting of the nucleotide sequence of SEQ ID NO: 10, or a sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 10.

TPO2 (Systematic name: YGR138C) refers to a protein in S. cerevisiae comprising, consisting essentially of, or consisting of the amino acid sequence of SEQ ID NO: 11, or a sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity or similarity with SEQ ID NO: 11. It is encoded by a gene comprising, consisting essentially of, or consisting of the nucleotide sequence of SEQ ID NO: 12, or a sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 12.

TPO3 (Systematic name: YPR156C) refers to a protein in S. cerevisiae comprising, consisting essentially of, or consisting of the amino acid sequence of SEQ ID NO: 13, or a sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity or similarity with SEQ ID NO: 13. It is encoded by a gene comprising, consisting essentially of, or consisting of the nucleotide sequence of SEQ ID NO: 14, or a sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 14.

TPO4 (Systematic name: YOR273C) refers to a protein in S. cerevisiae comprising, consisting essentially of, or consisting of the amino acid sequence of SEQ ID NO: 15, or a sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity or similarity with SEQ ID NO: 15. It is encoded by a gene comprising, consisting essentially of, or consisting of the nucleotide sequence of SEQ ID NO: 16, or a sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 16.

FLR1 (Systematic name: YBR008C) refers to a protein in S. cerevisiae comprising, consisting essentially of, or consisting of the amino acid sequence of SEQ ID NO: 17, or a sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity or similarity with SEQ ID NO: 17. It is encoded by a gene comprising, consisting essentially of, or consisting of the nucleotide sequence of SEQ ID NO: 18, or a sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 18.

YHK8 (Systematic name: YHR048W) refers to a protein in S. cerevisiae comprising, consisting essentially of, or consisting of the amino acid sequence of SEQ ID NO: 19, or a sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity or similarity with SEQ ID NO: 19. It is encoded by a gene comprising, consisting essentially of, or consisting of the nucleotide sequence of SEQ ID NO: 20, or a sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 20.

DTR1 (Systematic name: YBR180W) refers to a protein in S. cerevisiae comprising, consisting essentially of, or consisting of the amino acid sequence of SEQ ID NO: 21, or a sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity or similarity with SEQ ID NO: 21. It is encoded by a gene comprising, consisting essentially of, or consisting of the nucleotide sequence of SEQ ID NO: 22, or a sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 22.

HOL1 (Systematic name: YNR055C) refers to a protein in S. cerevisiae comprising, consisting essentially of, or consisting of the amino acid sequence of SEQ ID NO: 23, or a sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity or similarity with SEQ ID NO: 23. It is encoded by a gene comprising, consisting essentially of, or consisting of the nucleotide sequence of SEQ ID NO: 24, or a sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 24.

Accordingly, in some embodiments, the one or more membrane transporters of the DHA1 family may be encoded by a nucleotide sequence comprising, consisting essentially of or consisting of a sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with any of the nucleotide sequences set forth in SEQ ID NOs: 2, 4, 6, 8, 10, 12, 14, 16, 18, 20, 22 and 24; or comprise, consist essentially of or consist of an amino acid sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity or similarity with any of the amino acid sequences set forth in SEQ ID NOs: 1, 3, 5, 7, 9, 11, 13, 15, 17, 19, 21 and 23.

Similarly, according to some other embodiments, the one or more nucleotide sequences encoding the one ore more membrane transporters of the DHA1 family may be selected from a nucleotide sequence comprising, consisting essentially of or consisting of a sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with any of the nucleotide sequences set forth in SEQ ID NOs: 2, 4, 6, 8, 10, 12, 14, 16, 18, 20, 22 and 24.

In some embodiments, the one ore more membrane transporters of the DHA1 family may be encoded by a nucleotide sequence selected from the group consisting of:
(a) a nucleotide sequence encoding a polypeptide comprising an amino acid sequence that has at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity or similarity with the amino acid sequence of SEQ ID NOs: 1, 3, 5, 7, 9, 11, 13, 15, 17, 19, 21 and 23;
(b) a nucleotide sequence that has at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleotide sequence of SEQ ID NOs: 2, 4, 6, 8, 10, 12, 14, 16, 18, 20, 22 and 24; and
(c) a nucleotide sequence the sequence of which differs from the sequence of a nucleotide sequence of (a) or (b) due to the degeneracy of the genetic code.

The DHA1 transporters are well-conserved across yeast species. Accordingly, throughout this disclosure, the terms “AQR1”, “QDR1”, “QDR2”, “QDR3”, “TPO1”, “TPO2”, “TPO3”, “TPO4”, “FLR1”, “YHK8”, “DTR1”, “HOL1” also encompass any homologue protein and/or gene in any other yeast strain. As used herein, homologues of “AQR1”, “QDR1”, “QDR2”, “QDR3”, “TPO1”, “TPO2”, “TPO3”, “TPO4”, “FLR1”, “YHK8”, “DTR1”, “HOL1” may be understood to refer to a protein and/or gene characterized by a certain degree of sequence identity or similarity, for example at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity or similarity. In some embodiments, homologues of “AQR1”, “QDR1”, “QDR2”, “QDR3”, “TPO1”, “TPO2”, “TPO3”, “TPO4”, “FLR1”, “YHK8”, “DTR1”, “HOL1” may be understood to refer to proteins and/or genes characterized by a similar or identical function, such as causing a detectable excretion of amino acids in a yeast cell.

Amino acid excretion may be assessed by any suitable means known to the skilled person including high performance liquid chromatography (HPLC), ultra performance liquid chromatography (UPLC), gas chromatography-mass spectrometry (GC-MS), liquid chromatography-mass spectrometry (LC-MS) and other techniques including crossfeeding of amino acid auxotroph microorganisms in solid or liquid media, for example as described in Grenson, 1973 or Velasco et al., 2004, both incorporated herein by reference.

In some embodiments, the one or more membrane transporters of the DHA1 family as described herein is a single membrane transporter of the DHA1 family. Reduced expression of a single DHA1 member, such as by means of gene deletion, inactivation or downregulation of expression, preferably deletion or inactivation, more preferably deletion, could be a good compromise between reduced contamination of LAB and a potential trade-off phenotype such as reduced growth. Accordingly, in some embodiments, the one or more membrane transporters of the DHA1 family as described herein is a single membrane transporter AQR1 or a homologue thereof. In some embodiments, the one or more membrane transporters of the DHA1 family as described herein is a single membrane transporter QDR1 or a homologue thereof. In some embodiments, the one or more membrane transporters of the DHA1 family as described herein is a single membrane transporter QDR2 or a homologue thereof. In some embodiments, the one or more membrane transporters of the DHA1 family as described herein is a single membrane transporter QDR3 or a homologue thereof. In some embodiments, the one or more membrane transporters of the DHA1 family as described herein is a single membrane transporter TPO1 or a homologue thereof. In some embodiments, the one or more membrane transporters of the DHA1 family as described herein is a single membrane transporter TPO2 or a homologue thereof. In some embodiments, the one or more membrane transporters of the DHA1 family as described herein is a single membrane transporter TPO3 or a homologue thereof. In some embodiments, the one or more membrane transporters of the DHA1 family as described herein is a single membrane transporter TPO4 or a homologue thereof. In some embodiments, the one or more membrane transporters of the DHA1 family as described herein is a single membrane transporter FLR1 or a homologue thereof. In some embodiments, the one or more membrane transporters of the DHA1 family as described herein is a single membrane transporter YHK8 or a homologue thereof. In some embodiments, the one or more membrane transporters of the DHA1 family as described herein is a single membrane transporter DTR1 or a homologue thereof. In some embodiments, the one or more membrane transporters of the DHA1 family as described herein is a single membrane transporter HOL1 or a homologue thereof.

In some embodiments, there is provided a yeast strain as described herein having reduced expression of at least two, at least three, at least four, at least five, at least six, or at least seven membrane transporters of the DHA1 family relative to the expression level in a wild type strain or in a parental strain from which it is derived, or relative to a control or reference strain. In some embodiments, at least two membrane transporters of the DHA1 family show reduced expression relative to the expression level in a wild type strain or in a parental strain from which it is derived, or relative to a control or reference strain. In some embodiments, at least three membrane transporters of the DHA1 family show reduced expression relative to the expression level in a wild type strain or in a parental strain from which it is derived, or relative to a control or reference strain. In a preferred embodiment, the at least three membrane transporters comprise AQR1, QDR2 and QDR3 or homologues thereof, or QDR1, QDR2 and QDR3, or homologues thereof. In a preferred embodiment, at least four membrane transporters of the DHA1 family show reduced expression relative to the expression level in a wild type strain or in a parental strain from which it is derived, or relative to a control or reference strain. In a preferred embodiment, the at least four membrane transporters comprise AQR1, QDR2, QDR1 and TPO4, or homologues thereof. In some embodiments, at least five membrane transporters of the DHA1 family show reduced expression relative to the expression level in a wild type strain or in a parental strain from which it is derived, or relative to a control or reference strain. In some embodiments, at least six membrane transporters of the DHA1 family show reduced expression relative to the expression level in a wild type strain or in a parental strain from which it is derived, or relative to a control or reference strain. In a preferred embodiment, at least seven membrane transporters of the DHA1 family show reduced expression relative to the expression level in a wild type strain or in a parental strain from which it is derived, or relative to a control or reference strain. In a preferred embodiment, the at least seven membrane transporters comprise AQR1, QDR3, QDR2, QDR1, DTR1, HOL1 and TPO1, or homologues thereof.

In preferred embodiments, there is provided a yeast strain as described herein having reduced activity or expression of at least two or at least three membrane transporters of the DHA1 family selected from the group consisting of AQR1, QDR1, QDR2, QDR3, TPO1, TPO4, DTR1, HOL1 and/or (functional) homologues thereof, preferably AQR1, QDR1, QDR2, QDR3 and/or (functional) homologues thereof.

Throughout this disclosure, a yeast strain as described herein may relate to a variety of yeast species and strains. In some embodiments, a yeast strain as described herein may be a baker's yeast or a brewer's yeast, preferably a brewer's yeast. Exemplary yeast with industrially applicable characteristics, which can be used in accordance with embodiments herein include, but are not limited to Saccharomyces species (for example, Saccharomyces cerevisiae, Saccharomyces bayanus, Saccharomyces boulardii), Candida species (for example, Candida utilis, Candida krusei), Schizosaccharomyces species (for example Schizosaccharomyces pombe, Schizosaccharomyces japonicus), Pichia or Hansenula species (for example, Pichia pastoris or Hansenula polymorpha) species, and Brettanomyces species (for example, Brettanomyces claussenii).

In some embodiments, a yeast strain as described herein may be a so-called conventional yeast or a so-called non-conventional yeast, preferably a conventional yeast. Conventional yeasts typically include Saccharomyces cerevisiae and Schizosaccharomyces pombe. Non-conventional yeasts include at least Hansenula polymorpha, Yarrowia lipolytica, Pichia pastoris. Komagataella phaffii, Kluyveromyces lactis, Scheffersomyces stipitis, Dekkera bruxellensis, Brettanomyces claussenii, Candida albicans, C. glabrata, C. utilis and C. krusei, Pichia pastoris, Hansenula polymorpha.

Accordingly, in some embodiments, a yeast strain as described herein may belong to a genus selected from the group consisting of Saccharomyces, Schizosaccharomyces, Candida, Brettanomyces, Yarrowia, Hansenula, Dekkera, Kluyveromyces and Komagataella, preferably Saccharomyces. In some embodiments, a yeast strain as described herein may be species selected from the group consisting of Saccharomyces cerevisiae, Saccharomyces bayanus, Saccharomyces boulardii, Candida utilis, Candida krusei, Schizosaccharomyces pombe, Schizosaccharomyces japonicus, Brettanomyces claussenii, Yarrowia lipolytica, Pichia pastoris. Komagataella phaffii, Kluyveromyces lactis, Scheffersomyces stipitis, Dekkera bruxellensis, Candida albicans, Candida glabrata and Hansenula polymorpha, preferably Saccharomyces cerevisiae. In some embodiments, a yeast strain as described herein may be a Saccharomyces species, preferably Saccharomyces cerevisiae.

In some embodiments, a yeast strain as described herein may be an industrial yeast strain. Industrial yeast strains are known to allow robust growth and production under adverse industrial conditions. In some embodiments, an industrial yeast strain is an industrial Saccharomyces cerevisiae strain, including but not limited to Ethanol Red® (Fermentis), Fermiol® (DSM) and Thermosacc® (Lallemand). A preferred industrial yeast strain is Ethanol Red®.

Yeast strains as described herein show reduced excretion of amino acids. In some embodiments, amino acid excretion may be assessed by any suitable means known to the skilled person including high performance liquid chromatography (HPLC), ultra performance liquid chromatography (UPLC), gas chromatography-mass spectrometry (GC-MS), liquid chromatography-mass spectrometry (LC-MS) and other techniques including crossfeeding of amino acid auxotroph microorganisms in solid or liquid media, for example as described in Grenson, 1973 or Velasco et al., 2004, both incorporated herein by reference.

Accordingly, in some embodiments, there is provided a yeast strain as described herein, wherein the yeast strain shows reduced excretion of amino acids relative to a wild type strain or to a parental strain from which it is derived, or relative to a control or reference strain. In some embodiments, excretion of amino acids is reduced by 10%, 20%, 30%, 40%, 50%, 60%, 70%, 80%, 90%, 95%, 98%, 99% or 100% relative to the excretion level in a wild type strain or in a parental strain from which it is derived, or relative to a control or reference strain.

In some embodiments, the excretion of amino acids is measured during exponential growth. In some embodiments, the excretion of amino acids is measured during stationary phase. In a preferred embodiment, the excretion of amino acids may be measured during the diauxic shift. The excretion of amino acids may be measured in any suitable culture medium as described elsewhere herein.

In any aspect and/or embodiment described herein, a wild type strain or a parental strain or a reference strain or a control strain may belong to a genus selected from the group consisting of Saccharomyces, Schizosaccharomyces, Candida, Brettanomyces, Yarrowia, Hansenula, Dekkera, Kluyveromyces and Komagataella, preferably Saccharomyces. In some embodiments, a wild type strain or a parental strain as described herein may be species selected from the group consisting of Saccharomyces cerevisiae, Saccharomyces bayanus, Saccharomyces boulardii, Candida utilis, Candida krusei, Schizosaccharomyces pombe, Schizosaccharomyces japonicus, Brettanomyces claussenii, Yarrowia lipolytica, Pichia pastoris. Komagataella phaffii, Kluyveromyces lactis, Scheffersomyces stipitis, Dekkera bruxellensis, Candida albicans, Candida glabrata and Hansenula polymorpha, preferably Saccharomyces cerevisiae.

In any aspect and/or embodiment described herein, a wild type strain or a parental strain or a reference strain or a control strain may be an industrial yeast strain. Industrial yeast strains are known to allow robust growth and production under adverse industrial conditions. In some embodiments, an industrial yeast strain is an industrial Saccharomyces cerevisiae strain, including but not limited to Ethanol Red® (Fermentis) Fermiol® (DSM) and Thermosacc® (Lallemand). A preferred industrial yeast strain is Ethanol Red®.

Preferred wild type strains or parental strains or reference strains or control strains according to any aspect and/or embodiment described herein may be selected from the group consisting of Saccharomyces cerevisiae S288C (deposited as ATCC 204508), sigma1278b, CEN.PK (also known as CEN.PK2) (EUROSCARF:30000D), JK9-3D, SK1 (ATCC:204722), Y55, RM11-1a, D273-10B (ATCC:24657), FL100 (ATCC:28383), SEY6210 (ATCC:96099), W303 (ATCC:200060), X2180-1A (ATCC:204504), BY4743 (ATCC:201390). A preferred wild type strain or parental strain or reference strain or control strain is Saccharomyces cerevisiae S288C. Another preferred wild type strain or parental strain or reference strain or control strain is Saccharomyces cerevisiae sigma1278b.

B. Compositions

In a second aspect there is provided a composition comprising, consisting essentially of, or consisting of a yeast strain as described herein; and optionally one or more formulation excipients. Well-known non-limiting examples of suitable formulation excipients are citric acid esters of glycerol-monostearate or sorbitan monostearate as described in Reed, G. and Nagodawithana, T. W. (1991) Yeast Technology, 2nd ed., Van Nostrand Reinhold, New York, incorporated herein by reference.

In some embodiments, a yeast composition as described herein may further comprise a preservative. A non-limiting example of a suitable preservative is ascorbic acid.

Yeast compositions are available in a number of different forms which mainly differ with regard to their moisture contents. Accordingly, in some embodiments, there is provided a composition comprising, consisting essentially of, or consisting of a yeast strain as described herein, wherein the composition is a liquid yeast composition, a cream yeast composition, a compressed yeast or cake yeast composition, a dry yeast composition, or an instant yeast composition. In a preferred embodiment a yeast composition as described herein is a cream yeast composition, Dry yeast forms are generally a good choice for long-term storage, as they can be kept for over a year at room temperature without significant loss of viability. In a preferred embodiment, a yeast composition as described herein is a dry yeast composition or an instant yeast composition.

C. Methods

In a third aspect, there is provided a method for the production of yeast cells, comprising culturing a yeast strain as described herein in a suitable culture medium.

In a fourth aspect, there is provided a method for producing a fermentation product, comprising culturing a yeast strain as described herein on a fermentable substrate; and, optionally, recovering the fermentation product. In some embodiments, recovering the fermentation product may be purification of the fermentation product from the culture medium. In some embodiments, purification may be carried out by distillation.

A culturing step as described herein is not particularly limited. In some embodiments, culturing may involve batch culture, fed-batch culture or continuous culture, preferably batch or fed-batch culture.

In some embodiments of a method for producing a fermentation product as described herein, the fermentation substrate comprises, consists essentially of or consists of an unrefined or low-processed source of sugars. In some embodiments, the fermentation substrate comprises glucose and optionally one or more pentose sugars, e.g, arabinose and/or xylose. In an embodiment, the fermentation substrate may comprise lignocellulosic biomass or a hydrolysate thereof.

In some embodiments, the fermentation product comprises an industrially useful molecule, for example a carbohydrate, a lipid, an organic molecule, a nutrient, a fertilizer, a biofuel, a cosmetic (or precursor thereof), a pharmaceutical or biopharmaceutical product (or precursor thereof), or two or more of any of the listed items. In some embodiments of a method for producing a fermentation product as described herein, the fermentation product is an alcohol, preferably a lower alkyl alcohol such as ethanol, propanol or butanol, more preferably ethanol.

In a fifth aspect there is provided a method for the production of a product of interest, comprising culturing a yeast cell as described herein in a suitable culture medium. In some embodiments, there is provided a method for the production of a product of interest, comprising culturing a yeast cell as described herein in a suitable culture medium, wherein said yeast cell comprises a vector encoding the product of interest or encoding one or more enzymes capable of producing the product of interest. In some embodiments, there is provided a method for the production of a product of interest, comprising:
a) optionally, transforming a yeast cell as described herein with a vector encoding the product of interest or encoding one or more enzymes capable of producing the product of interest;
b) culturing a yeast cell as described herein in a culture medium to produce the product of interest; and
c) optionally, isolating the product of interest from the yeast cell or culture medium.

In some embodiments, the product of interest is a small molecule, for example a small-molecule drug. In some embodiments, the vector may comprise the complete metabolic pathway required for producing the product of interest, such as the complete metabolic pathway required for producing the small molecule, for example a small-molecule drug.

In some embodiments, the product of interest is a protein. In some embodiments, the vector is an expression vector comprising a gene encoding the protein. Accordingly, in some embodiments there is provided a method for the production of a protein, comprising culturing a yeast cell as described herein in a suitable culture medium, wherein said yeast cell comprises an expression vector comprising a gene encoding the protein. In some embodiments, the gene encoding the protein is stably integrated in the genome.

In some embodiments, there is provided a method for the production of a protein, comprising
a) optionally, transforming a yeast cell as described herein with an expression vector comprising a gene encoding the protein;
b) culturing a yeast cell as described herein in a suitable culture medium under conditions which allow expression of the protein; and
c) optionally, isolating the protein from the yeast cell or culture medium.

In some embodiments, the protein may be a recombinant protein or a native protein. In a preferred embodiment, the protein may be a recombinant protein. As used herein, a recombinant protein may refer to any protein which is produced from a recombinant DNA sequence. As used herein, a native protein is a protein that is naturally expressed in the yeast cell that is cultured.

Methods for yeast transformation are known to a skilled person and are for example described in “Methods in Yeast Genetics and Genomics, 2015 Edition: A CSHL Course Manual, Cold Spring Harbor Laboratory Press, by Maitreya Dunham, Marc Gartenberg and Grant W. Brown”, incorporated herein by reference.

In some embodiments, there is provided a method for the production of yeast cells as described herein and/or a method for producing a fermentation product as described herein and/or a method for the production of a product of interest as described herein, wherein contamination of lactic acid bacteria is eliminated, reduced or prevented.

In some embodiments, there is provided a method for the production of yeast cells as described herein and/or a method for producing a fermentation product as described herein and/or a method for the production of a product of interest as described herein, wherein contamination of lactic acid bacteria is eliminated or prevented.

In some embodiments, there is provided a method for the production of yeast cells as described herein and/or a method for producing a fermentation product as described herein and/or a method for the production of a product of interest as described herein, wherein contamination of lactic acid bacteria is reduced.

In some embodiments, eliminated or prevented means that no lactic acid bacteria are present at a detectable level. A detectable level may be a detectable level when measured by sequencing-based, PCR-based, imaging-based, flow cytometry-based and/or plating-based methods. In some embodiments, a detectable level may mean 1000, 100, 10 or 1 CFU per ml of culture medium.

In some embodiments, reduced may mean a reduction to a level below 10⁶, 10⁵, 10⁴, 1000, 100, 10 or 1 CFU per ml of culture medium. In some embodiments, reduction may mean a reduction to an undetectable level, such as below 1000, 100, 10 or 1 CFU per ml of culture medium.

In some embodiments, the elimination, reduction and/or prevention of contamination of lactic acid bacteria may be assessed after 1 hour, 2 hours, 4 hours, 6 hours, 8 hours, 10 hours, 12 hours, 16 hours, 20 hours, 24 hours, 36 hours or 48 hours of culturing the yeast strain.

As used herein, “lactic acid bacteria” or “LAB” refers to any bacterium producing lactic acid as a major metabolic end product of carbohydrate fermentation. Lactic acid bacteria include at least all bacteria of the order Lactobacillales. The order Lactobacillales includes at least the families Aerococcaceae, Carnobacteriaceae, Enterococcaceae, Lactobacillaceae, Leuconostocaceae and Streptococcaceae. The order Lactobacillales includes at least the genera Lactobacillus, Leuconostoc, Pediococcus, Lactococcus, Streptococcus, Abiotrophia, Aerococcus, Aerosphaera, Agitococcus, Alkalibacterium, Allofustis, Alloiococcus, Atopobacter, Atopococcus, Atopostipes, Bavariicoccus, Carnobacterium, Carnococcus, Catellicoccus, Chungangia, Convivina, Desemzia, Dolosicoccus, Dolosigranulum, Enterococcus, Eremococcus, Facklamia, Floricoccus, Fructobacillus, Globicatella, Granulicatella, Ignavigranum, Isobaculum, Jeotgalibaca, Lacticigenium, Lactovum, Marinilactibacillus, Melissococcus, Oenococcus, Okadaella, Pilibacter, Pisciglobus, Sharpea, Sporolactobacillus, Tetragenococcus, Trichococcus, Vagococcus and Weissella. Generally, but non-limiting, LAB are gram-positive, low-GC, acid-tolerant, nonsporulating, nonrespiring, and either rod-shaped (bacilli) or spherical (cocci) bacteria. According to any embodiment described herein, the lactic acid bacteria may comprise a Lactobacillus species. In some embodiments, the Lactobacillus species is L. fermentum.

D. Uses

In a sixth aspect, there is provided use of a yeast strain and/or of a composition as described herein for eliminating, reducing or preventing contamination of lactic acid bacteria in yeast cultures.

In some embodiments, there is provided a use of a yeast strain and/or of a composition as described herein for eliminating or preventing contamination of lactic acid bacteria in yeast cultures.

In some embodiments, there is provided a use of a yeast strain and/or of a composition as described herein for reducing contamination of lactic acid bacteria in yeast cultures.

In some embodiments, eliminated or prevented means that no lactic acid bacteria are present at a detectable level. A detectable level may be a detectable level when measured by sequencing-based, PCR-based, imaging-based, flow cytometry-based and/or plating-based methods. In some embodiments, a detectable level may mean 1000, 100, 10 or 1 CFU per ml of culture medium.

In some embodiments, reduced may mean a reduction to a level below 10⁶, 10⁵, 10⁴, 1000, 100, 10 or 1 CFU per ml of culture medium. In some embodiments, reduction may mean a reduction to an undetectable level, such as below 1000, 100, 10 or 1 CFU per ml of culture medium.

Also provided is a method for eliminating, reducing or preventing contamination of lactic acid bacteria in yeast cultures, comprising culturing a yeast strain as described herein.

Throughout this disclosure and particularly in the context of methods and uses as described herein, contamination of lactic acid bacteria can be assessed by any suitable means as known to the skilled person, including sequencing-based, PCR-based, imaging-based, flow cytometry-based and plating-based methods. Contamination of lactic acid bacteria can also be assessed by detection of lactic acid.

According to any embodiment described herein, the lactic acid bacteria may comprise a Lactobacillus species. In some embodiments, the Lactobacillus species is L. fermentum.

Throughout this disclosure and particularly in the context of yeast strains, compositions, methods and uses as described herein, there is no particular limitation on a suitable culture medium. Microbial culture environments can comprise a wide variety of culture media. The selection of a particular culture medium can depend upon the desired application. Growth conditions depend not only on the chemical composition of a culture medium but also on other parameters including temperature, amounts of light, pH, CO₂ levels, and the like. In some embodiments, a suitable culture medium may be a rich medium or a minimal medium. A suitable culture medium may be a complex medium or a defined medium. In a preferred embodiment, the culture medium is a defined medium. Typically, a suitable culture medium contains a carbon source, a nitrogen source and inorganic salts, and is capable of supporting the growth of the yeast.

In some embodiments, the culture medium does not contain free amino acids. In some embodiments, the culture medium has a low level of free amino acids, such as less than 1 g/l, less than 500 mg/l, less than 250 mg/l, less than 100 mg/l, less than 50 mg/l, less than 25 mg/l, less than 10 mg/l, less than 5 mg/l, less than 2.5 mg/l, less than 1 mg/l, less than 0.5 mg/l, less than 0.25 mg/l or less than 0.1 mg/l.

In some embodiments, there is provided a method for the production of yeast cells as described herein and/or a method for producing a fermentation product and/or a method for producing a product of interest as described herein, wherein contamination of lactic acid bacteria is eliminated, reduced or prevented.

In some embodiments according to any of the methods and uses described herein, the yeast strain as described herein shows reduced activity of one or more membrane transporters of the DHA1 family relative to a wild type strain or to a parental strain from which it is derived. In some embodiments reduced activity, for example as obtained by not inducing or repressing a promoter as described herein, may occur during one or more specific phases or time frames of the culturing step. In some embodiments, a promoter as described herein, such as an inducible promoter, can be induced during one or more specific phases or time frames of the culturing step. In some embodiments, a promoter as described herein, such as an repressible promoter, can be repressed during one or more specific phases or time frames of the culturing step. The one or more specific phases or time frames are preferably chosen so that the activity of the one ore more DHA1 transporters is reduced when the likelihood for LAB contamination is highest. In some embodiments, a specific phase or timeframe may include or exclude the first 1, 2, 3, 4, 5, 6, 7, 8, 9 or 10 hours of culturing. In some embodiments, a specific phase or timeframe may include or exclude the timeframe wherein the density of the yeast strain as described herein in the culture medium is below 1000, 10⁴, 10⁵, 10⁶, 10⁷ or 10⁸ CFU/ml.

General Information

Unless stated otherwise, all technical and scientific terms used herein have the same meaning as customarily and ordinarily understood by a person of ordinary skill in the art to which this invention belongs, and read in view of this disclosure.

As used herein, the term “promoter” or “regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” and/or “repressible” promoter is a promoter that is physiologically or developmentally regulated to be induced and/or repressed, e.g. by the application of a chemical inducer or repressing signal.

As used herein, the term “operably linked” refers to a linkage of polynucleotide elements in a functional relationship. A nucleic acid is “operably linked” when it is placed into a functional relationship with another nucleic acid sequence. For instance, a transcription regulatory sequence such as a promoter is operably linked to a coding sequence if it affects the transcription of the coding sequence. Operably linked means that the DNA sequences being linked are typically contiguous and, where necessary to join two protein encoding regions, contiguous and in reading frame.

As used herein, a “regulator” or “transcriptional regulator” is a protein that controls the rate of transcription of genetic information from DNA to messenger RNA, by binding to a specific DNA sequence.

The terms “protein” or “polypeptide” are used interchangeably and refer to molecules consisting of a chain of amino acids, without reference to a specific mode of action, size, 3-dimensional structure or origin.

The term “gene” means a DNA fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence, a coding region and a 3′-nontranslated sequence (3′-end) e.g. comprising a polyadenylation- and/or transcription termination site.

“Expression of a gene” refers to the process wherein a DNA region which is operably linked to appropriate regulatory regions, particularly a promoter, is transcribed into an RNA, which is biologically active, i.e. which is capable of being translated into a biologically active protein or peptide.

In amino acid sequences as described herein, amino acids or “residues” are denoted by three-letter symbols. These three-letter symbols as well as the corresponding one-letter symbols are well known to the person skilled in the art and have the following meaning: A (Ala) is alanine, C (Cys) is cysteine, D (Asp) is aspartic acid, E (Glu) is glutamic acid, F (Phe) is phenylalanine, G (Gly) is glycine, H (His) is histidine, I (Ile) is isoleucine, K (Lys) is lysine, L (Leu) is leucine, M (Met) is methionine, N (Asn) is asparagine, P (Pro) is proline, Q (Gin) is glutamine, R (Arg) is arginine, S (Ser) is serine, T (Thr) is threonine, V (Val) is valine, W (Trp) is tryptophan, Y (Tyr) is tyrosine. A residue may be any proteinogenic amino acid, but also any non-proteinogenic amino acid such as D-amino acids and modified amino acids formed by post-translational modifications, and also any non-natural amino acid, as described herein.

Sequence Identity

It is to be understood that each nucleic acid molecule or protein fragment or polypeptide or peptide or derived peptide or construct as identified herein by a given sequence identity number (SEQ ID NO) is not limited to this specific sequence as disclosed. Each coding sequence as identified herein encodes a given protein fragment or polypeptide or peptide or derived peptide or construct or is itself a protein fragment or polypeptide or construct or peptide or derived peptide. Throughout this application, each time one refers to a specific nucleotide sequence SEQ ID NO (take SEQ ID NO: X as example) encoding a given protein fragment or polypeptide or peptide or derived peptide, one may replace it by:
i. a nucleotide sequence comprising a nucleotide sequence that has at least 60% sequence identity with SEQ ID NO: X;
ii. a nucleotide sequence the sequence of which differs from the sequence of a nucleic acid molecule of (i) due to the degeneracy of the genetic code; or
iii. a nucleotide sequence that encodes an amino acid sequence that has at least 60% amino acid identity or similarity with an amino acid sequence encoded by a nucleotide sequence SEQ ID NO: X.

Throughout this application, each time one refers to a specific amino acid sequence SEQ ID NO (take SEQ ID NO: Y as example), one may replace it by: a polypeptide comprising an amino acid sequence that has at least 60% sequence identity or similarity with amino acid sequence SEQ ID NO: Y. Another preferred level of sequence identity or similarity is 70%. Another preferred level of sequence identity or similarity is 80%. Another preferred level of sequence identity or similarity is 90%. Another preferred level of sequence identity or similarity is 95%. Another preferred level of sequence identity or similarity is 99%.

Each nucleotide sequence or amino acid sequence described herein by virtue of its identity or similarity percentage with a given nucleotide sequence or amino acid sequence respectively has in a further preferred embodiment an identity or a similarity of at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% with the given nucleotide or amino acid sequence, respectively.

The terms “homology”, “sequence identity” and the like are used interchangeably herein. Sequence identity is described herein as a relationship between two or more amino acid (polypeptide or protein) sequences or two or more nucleic acid (polynucleotide) sequences, as determined by comparing the sequences. In a preferred embodiment, sequence identity is calculated based on the full length of two given SEQ ID NO's or on a part thereof. Part thereof preferably means at least 50%, 60%, 70%, 80%, 90%, or 100% of both SEQ ID NO's. In the art, “identity” also refers to the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in Bioinformatics and the Cell: Modern Computational Approaches in Genomics, Proteomics and transcriptomics, Xia X., Springer International Publishing, New York, 2018; and Bioinformatics: Sequence and Genome Analysis, Mount D., Cold Spring Harbor Laboratory Press, New York, 2004. “Sequence identity” and “sequence similarity” can be determined by alignment of two peptide or two nucleotide sequences using global or local alignment algorithms, depending on the length of the two sequences. Sequences of similar lengths are preferably aligned using a global alignment algorithm (e.g. Needleman-Wunsch) which aligns the sequences optimally over the entire length, while sequences of substantially different lengths are preferably aligned using a local alignment algorithm (e.g. Smith-Waterman). Sequences may then be referred to as “substantially identical” or “essentially similar” when they (when optimally aligned by for example the program EMBOSS needle or EMBOSS water using default parameters) share at least a certain minimal percentage of sequence identity (as described below).

A global alignment is suitably used to determine sequence identity when the two sequences have similar lengths. When sequences have a substantially different overall length, local alignments, such as those using the Smith-Waterman algorithm, are preferred. EMBOSS needle uses the Needleman-Wunsch global alignment algorithm to align two sequences over their entire length (full length), maximizing the number of matches and minimizing the number of gaps. EMBOSS water uses the Smith-Waterman local alignment algorithm. Generally, the EMBOSS needle and EMBOSS water default parameters are used, with a gap open penalty=10 (nucleotide sequences)/10 (proteins) and gap extension penalty=0.5 (nucleotide sequences)/0.5 (proteins). For nucleotide sequences the default scoring matrix used is DNAfull and for proteins the default scoring matrix is Blosum62 (Henikoff & Henikoff, 1992, PNAS 89, 915-919).

Alternatively percentage similarity or identity may be determined by searching against public databases, using algorithms such as FASTA, BLAST, etc. Thus, the nucleic acid and protein sequences of some embodiments of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to oxidoreductase nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., BLASTx and BLASTn) can be used. See the homepage of the National Center for Biotechnology Information accessible on the world wide web at www.ncbi.nlm.nih.gov/.

Optionally, in determining the degree of amino acid similarity, the skilled person may also take into account so-called conservative amino acid substitutions.

As used herein, “conservative” amino acid substitutions refer to the interchangeability of residues having similar side chains. Examples of classes of amino acid residues for conservative substitutions are given in the Tables below.

| | |
|---|---|
| Acidic Residues | Asp (D) and Glu (E) |
| Basic Residues | Lys (K), Arg (R), and His (H) |
| Hydrophilic Uncharged Residues | Ser (S), Thr (T), Asn (N), and |
| | Gln (Q) |
| Aliphatic Uncharged Residues | Gly (G), Ala (A), Val (V), |
| | Leu (L), and Ile (I) |
| Non-polar Uncharged Residues | Cys (C), Met (M), and Pro (P) |
| Aromatic Residues | Phe (F), Tyr (Y), and Trp (W) |

Alternative conservative amino acid residue substitution classes:

| | | | |
|---|---|---|---|
| 1 | A | S | T |
| 2 | D | E | |
| 3 | N | Q | |
| 4 | R | K | |
| 5 | I | L | M |
| 6 | F | Y | W |

Alternative physical and functional classifications of amino acid residues:

| | |
|---|---|
| Alcohol group-containing | S and T |
| residues | |
| Aliphatic residues | I, L, V, and M |
| Cycloalkenyl-associated | F, H, W, and Y |
| residues | |
| Hydrophobic residues | A, C, F, G, H, I, L, M, R, T, V, W, |
| | and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in | A, C, D, E, G, H, K, N, Q, R, S, |
| turn formation | P and T |
| Flexible residues | Q, T, K, S, G, P, D, E, and R |

Yeast

In addition to the variety of yeast species and strains that can be used in accordance with embodiments herein as described elsewhere, genetically modified variants, or synthetic yeast based on a “chassis” of a known species can be provided. Furthermore, fully synthetic microorganism genomes can be synthesized and transplanted into single microbial cells, to produce synthetic microorganisms capable of continuous self-replication (see Gibson et al. (2010), “Creation of a Bacterial Cell Controlled by a Chemically Synthesized Genome,” Science 329: 52-56, which is incorporated herein by reference). As such, in some embodiments, the microorganism is fully synthetic. A desired combination of genetic elements, including elements that regulate gene expression, and elements encoding gene products (for example immunity modulators, poison, antidote, and industrially useful molecules also called product of interest) can be assembled on a desired chassis into a partially or fully synthetic microorganism. Description of genetically engineered microbial organisms for industrial applications can also be found in Wright, et al. (2013) “Building-in biosafety for synthetic biology” Microbiology 159: 1221-1235, incorporated herein by reference.

Expression

Expression (i.e., gene expression) may be assessed by any suitable method known to a person of skill in the art. For example, expression may be assessed by measuring the levels of expression on the level of the mRNA or the protein by standard assays known to a person of skill in the art, such as qRT-PCR, RNA sequencing, Northern blot analysis, Western blot analysis, mass spectrometry analysis of protein-derived peptides or ELISA.

Expression may be assessed at any time after administration of the gene construct, expression vector or composition as described herein.

In this document and in its claims, the verb “to comprise” and its conjugations is used in its non-limiting sense to mean that items following the word are included, but items not specifically mentioned are not excluded. In addition, the verb “to consist” may be replaced by “to consist essentially of” meaning that a composition as described herein may comprise additional component(s) than the ones specifically identified, said additional component(s) not altering the unique characteristic of the invention. In addition, the verb “to consist” may be replaced by “to consist essentially of” meaning that a method as described herein may comprise additional step(s) than the ones specifically identified, said additional step(s) not altering the unique characteristic of the invention.

Reference to an element by the indefinite article “a” or “an” does not exclude the possibility that more than one of the element is present, unless the context clearly requires that there be one and only one of the elements. The indefinite article “a” or “an” thus usually means “at least one”.

As used herein, with “at least” a particular value means that particular value or more. For example, “at least 2” is understood to be the same as “2 or more” i.e., 2, 3, 4, 5, 6, 7, 8, 9, 10, 11, 12, 13, 14, 15, . . . , etc.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The word “about” or “approximately” when used in association with a numerical value (e.g. about 10) preferably means that the value may be the given value (of 10) more or less 0.1% of the value. As used herein, the term “and/or” indicates that one or more of the stated cases may occur, alone or in combination with at least one of the stated cases, up to with all of the stated cases.

Various embodiments are described herein. Each embodiment as identified herein may be combined together unless otherwise indicated.

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

One skilled in the art will recognize many methods and materials similar or equivalent to those described herein, which could be used in the practice of the present invention. Indeed, the present invention is in no way limited to the methods and materials described.

The present invention is further described by the following examples which should not be construed as limiting the scope of the invention.

### DESCRIPTION OF THE FIGURES

FIG. 1. Relative growth in co-cultures of L. fermentum (LAB) and S. cerevisiae (yeast) (wild-type and mutant with aqr1, qdr2, qdr1 and tpo4 deletions). The graph shows the mean of three independent experiments. The error bars represent standard deviations. Student's t-test, * P value=0.029.

FIG. 2. Relative growth in co-cultures of L. fermentum (LAB) and S. cerevisiae (yeast) (wild-type and mutant with aqr1, qdr3, qdr2, qdr1, dtr1, hol1 and tpo1 deletions). The graph shows the mean of three independent experiments. The error bars represent standard deviations. Student's t-test, ** P value=0.0096.

FIG. 3. Relative growth in co-cultures of L. fermentum (L.f.) and S. cerevisiae (wild-type (w-t), mutant with triple aqr1 qdr3 qdr2 deletions, and mutants with single aqr1, qdr3, or qdr2 deletions) (A). Cell densities of L. fermentum (estimated by counting the number of colony forming units (CFU) per ml of culture) just after inoculation (time 0) and 48 hours after incubation at 29° C. in the absence or presence of amino acids. The graph shows the mean of three to four independent experiments. The error bars represent standard deviations. (B) Left. Initial cell densities (number of colony forming units per ml of culture) of L. fermentum (black bars) and yeast (grey bars) just after inoculation of the co-cultures. Right. Cell densities of the co-cultures after 48 hours of incubation at 29° C. The graph shows the mean of four to five independent experiments. The error bars represent standard deviations. Student's t-test, ** P value=0.0014. (C) Propagation ratios of L. fermentum vs. yeast after 48 h of co-culture (calculated from data shown in B). Error bars represent standard deviations. Student's t-test, ** P value=0.0014. (D) Propagation ratios of L. fermentum vs. yeast (wild-type vs. mutants with single aqr1, qdr3, or qdr2 deletion) after 48 h of co-culture (experimental conditions as in B and C). Error bars represent standard deviations. Student's t-test (NS: not significant, p>0.05).

FIG. 4. Propagation ratios of L. fermentum and S. cerevisiae (wild-type Ethanol Red strain (w-t) vs. derivative mutant with qdr1, qdr2, and qdr3 deletions). All conditions as in FIG. 3B. The graph shows the mean of two independent experiments. Error bars represent standard deviations. Student's t-test, ** P value=0.0181.

### EXAMPLES

### Example 1: Reduced Expression of Four DHA1-Family Genes in Yeast Causes Reduced Amino Acid Excretion and Reduced Propensity to Support Growth of Cocultivated Lactic Acid Bacteria (LAB)

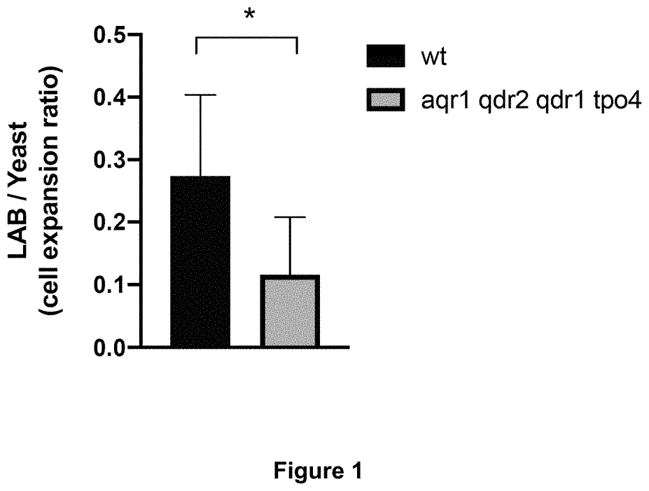

Yeast S. cerevisiae strains 23344c (wild-type, wt), GK097 (aqr1Δ qdr2Δ qdr1Δ tpo4Δ) were cocultivated with Lactobacillus fermentum (auxotroph for several amino acids) on a MES-buffered minimal glucose medium (169) devoid of amino acids and containing NH₄⁺ as sole nitrogen source. The number of cells per ml of culture was measured just after inoculation (time 0) and 64 h after growth in order to calculate the LAB to yeast cell expansion ratios. As can be seen in FIG. 1, growth of L. fermentum is significantly reduced when cocultivated with S. cerevisiae strain GK097 compared to cocultivation with S. cerevisiae strain 23344c, indicating reduced amino acid excretion by S. cerevisiae strain GK097.

### Example 2: Reduced Expression of Seven DHA1-Family Genes in Yeast Causes Reduced Amino Acid Excretion and Reduced Propensity to Support Growth of Cocultivated Lactic Acid Bacteria (LAB)

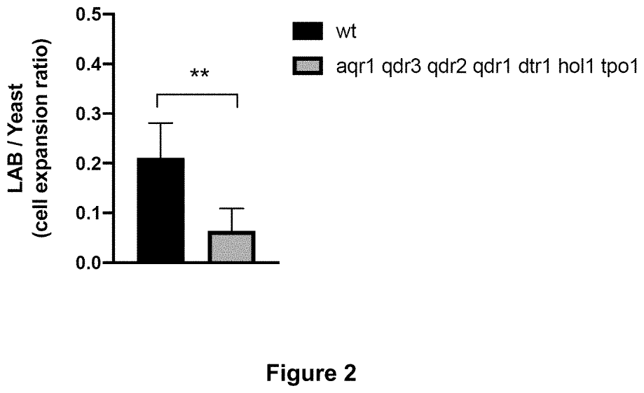

Yeast S. cerevisiae strains 23344c (wild-type, wt) and FV1170 (aqr1Δ qdr3Δ qdr2Δ qdr1Δ dtr1Δ hol1Δ tpo1Δ) were cocultivated with Lactobacillus fermentum (auxotroph for several amino acids) on a MES-buffered minimal glucose medium (169) devoid of amino acids and containing NH₄⁺ as sole nitrogen source. The number of cells per ml of culture was measured just after inoculation (time 0) and 64 h after growth in order to calculate the LAB to yeast cell expansion ratios. As can be seen in FIG. 2, growth of L. fermentum is significantly reduced when cocultivated with S. cerevisiae strain FV1170 compared to cocultivation with S. cerevisiae strain 23344c, indicating reduced amino acid excretion by S. cerevisiae strain FV1170.

### Example 3. Reduced Expression of Three DHA1-Family Genes in Yeast Causes Reduced Amino Acid Excretion and Reduced Propensity to Support Growth of Cocultivated Lactic Acid Bacteria

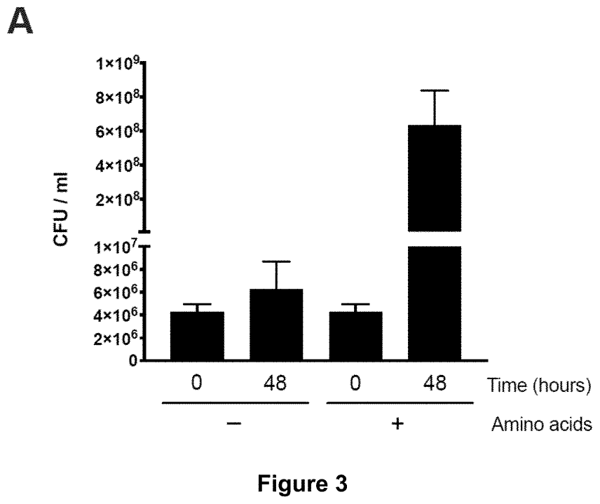

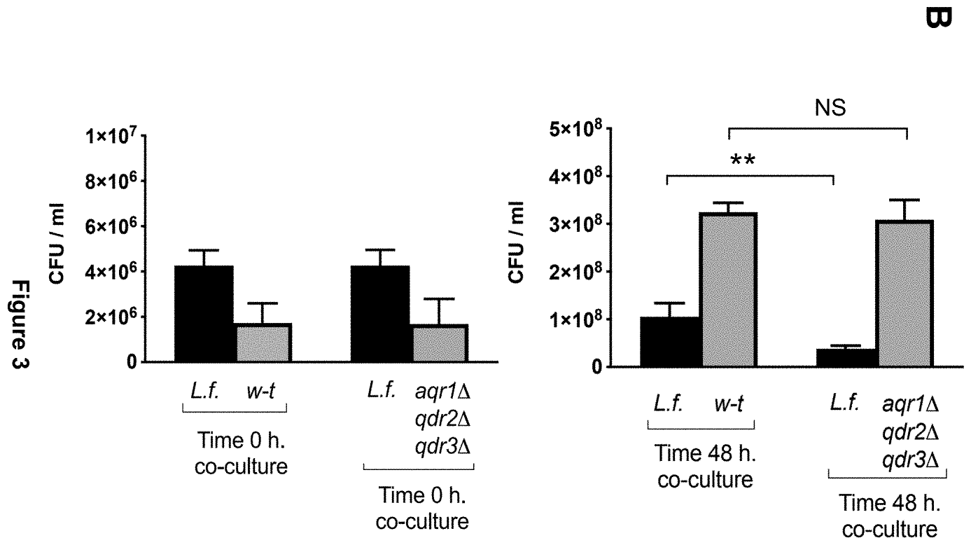

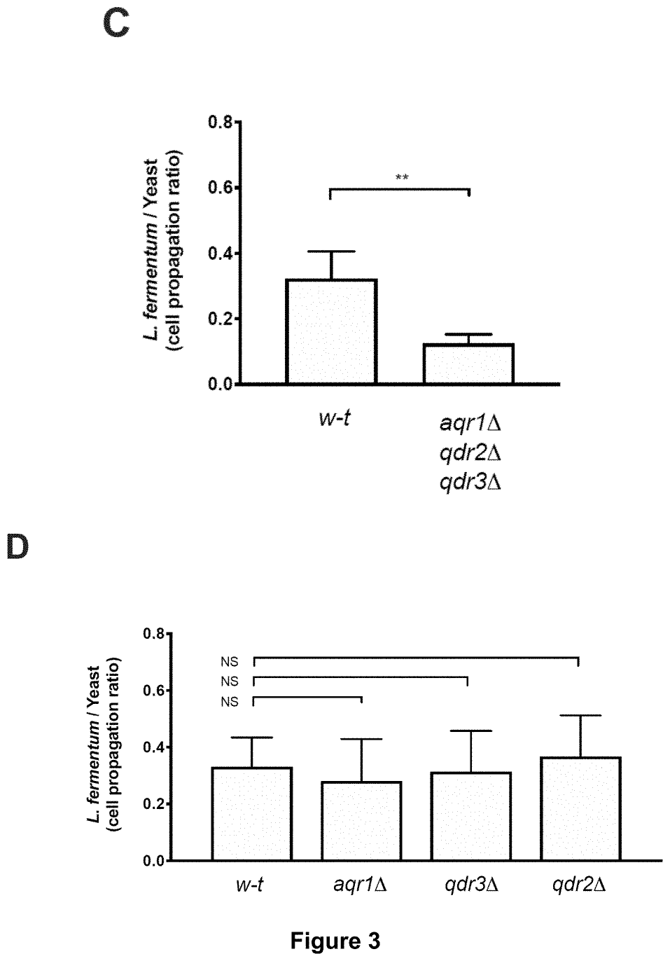

(A). The MES-buffered minimal glucose medium (169) containing NH₄⁺ as sole nitrogen source, and to which amino acids have been added or not (as amino acid dropout mixture), was inoculated with Lactobacillus fermentum (auxotroph for several amino acids). Samples of the cultures were withdrawn just after inoculation (time 0) and after 48 hours of incubation at 29° C. Cell densities were measured by counting the number of colony-forming units per ml of culture (CFU/ml). The results presented in FIG. 3A confirm that amino acids represent a limiting nutrient for propagation of the bacterium (which is auxotroph for several amino acids). (B). Yeast S. cerevisiae strains 23344c (wild-type, w-t) and GK121 (aqr1Δ qdr3Δ qdr2Δ) were co-cultivated with Lactobacillus fermentum on the MES-buffered minimal glucose medium (169) devoid of amino acids and containing NH₄⁺ as sole nitrogen source. The number of colony-forming units per ml of culture (CFU/ml) was measured just after inoculation (time 0) and 48 h after incubation at 29° C. The results illustrated in FIG. 3B show L. fermentum is capable of propagating when co-cultivated with S. cerevisiae, and that this propagation is significantly reduced when S. cerevisiae carries the aqr1Δ, qdr3Δ, and qdr2Δ mutations. (C). Data of (B) were used to calculate the L. fermentum to yeast propagation ratios. As can be seen in FIG. 3C, this propagation ratio is reduced by more than 50% when S. cerevisiae contains the aqr1Δ, qdr3Δ, and qdr2Δ mutations, indicating reduced amino acid excretion by S. cerevisiae strain GK122. (D). Yeast S. cerevisiae strains 23344c (wild-type, w-t), FV812 (aqr1Δ), CM005 (qdr3Δ), or GK089 (qdr2Δ) were co-cultivated with Lactobacillus fermentum as detailed in B. The data were used to calculate the L. fermentum to yeast propagation ratios. As can be seen in FIG. 3D, the propagation ratios were not significantly reduced when S. cerevisiae contains the single aqr1Δ, qdr3Δ, or qdr2Δ mutations.

### Example 4. Reduced Expression of Three DHA1-Family Genes in an Industrial Yeast Strain Causes Reduced Amino Acid Excretion and Reduced Propensity to Support Growth of Cocultivated Lactic Acid Bacteria

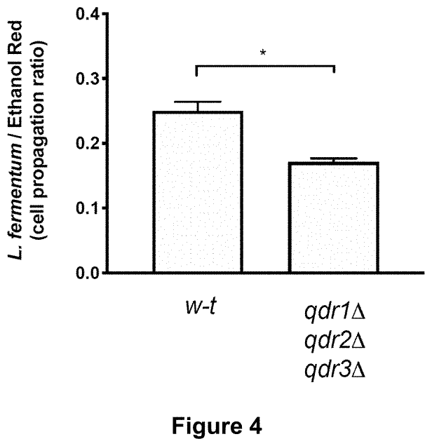

Industrial yeast S. cerevisiae strain Ethanol Red (wild-type, w-t) and the derivative strain CF171 (qdr1Δ qdr2Δ qdr3Δ) were cocultivated with Lactobacillus fermentum on a MES-buffered minimal glucose medium (169) devoid of amino acids and containing NH₄⁺ as sole nitrogen source. The number of colony forming units per ml of culture (CFU/ml) was measured just after inoculation (time 0) and 48 h after growth in order to calculate the L. fermentum to yeast cell propagation ratios. As can be seen in FIG. 4, growth of L. fermentum is significantly reduced when cocultivated with S. cerevisiae strain CF171 compared to cocultivation with the parental S. cerevisiae Ethanol Red strain, indicating reduced amino acid excretion by S. cerevisiae strain CF171.

Composition of the 169 Medium Used for Cocultures of S. cerevisiae and L. fermentum

The medium is prepared by mixing basal 169 medium with samples of trace metals (×1000) and vitamins (×100) solutions. One liter of basal 169 medium contains: 0.7 g MgSO₄.7 H₂O, 1 g KH₂PO₄, 0.4 g CaCl₂·2H₂O, 0.5 g NaCl, 5 g (NH₄)₂SO₄, 19.5 g 2-(N-morpholino)ethanesulfonic acid (MES). This medium was adjusted to pH 6.1 with NaOH 10M and sterilized. 100 ml of vitamin solution (×100) contains 15 μg D-biotin, 10 mg thiamine·HCl, 100 mg inositol, 20 mg calcium D-panthothenate, 10 mg pyridoxin·HCl, 5.6 mg folic acid, 9 mg nicotinic acid, 0.5 mg 4-aminobenzoic acid, 9 mg riboflavin, and 150 mg glutathione. 100 ml of trace metals solution (×1000) contains 10 mg H₃BO₄, 1 mg CuSO₄·5H₂O, 2 mg KI, 4 mg Na₂MoO₄·2H₂O, 14 mg ZnSO₄·7H₂O, 10 g citric acid·H₂O, 400 mg MnSO₄·H₂O, 5 g FeCl₃·6H₂O, and 190 mg CoCl₂·H₂O.

Methods for Isolating Yeast Mutants.

The genes in the above-described yeast mutants were deleted by either the classical PCR-based replacement with antibiotic resistance genes (Wach et al., 1994) or a recently described CRISPR/Cas9-based method (Mans et al., 2018).

### REFERENCES

Beckner, M., Ivey, M. L., & Phister, T. G. (2011). Microbial contamination of fuel ethanol fermentations. Letters in Applied Microbiology, 53(4), 387-394. http://doi.org/10.1111/j.1472-765X.2011.03124.x
Challinor, S. W., & Rose, A. H. (1954). Interrelationships between a yeast and a bacterium when growing together in defined medium. Nature, 174(4436), 877-878. http://doi.org/10.1038/174877b0
Grenson, M. (1973). Specificity and regulation of the uptake and retention of amino acids and pyrimidines in yeast. In Z. Vanek, Z. Hostalek, & J. Cudlin (Eds.), Genetics of industrial microorganisms (pp. 179-193). Prague: New York, Elsevier Pub. Co.
Mans, R., Wijsman, M., Daran-Lapujade, P., and Daran, J.-M. (2018). A protocol for introduction of multiple genetic modifications in Saccharomyces cerevisiae using CRISPR/Cas9. FEMS Yeast Res. 18, 3329.
Narendranath, N. V., Hynes, S. H., Thomas, K. C., & Ingledew, W. M. (1997). Effects of lactobacilli on yeast-catalyzed ethanol fermentations. Applied and Environmental Microbiology, 63(11),
Ponomarova, O., Gabrielli, N., Sévin, D. C., Mülleder, M., Zirngibl, K., Bulyha, K., et al. (2017). Yeast Creates a Niche for Symbiotic Lactic Acid Bacteria through Nitrogen Overflow. Cell Systems. http://doi.org/10.1016/j.cels.2017.09.002
Skinner, K. A., & Leathers, T. D. (2004). Bacterial contaminants of fuel ethanol production. Journal of Industrial Microbiology & Biotechnology, 31(9), 401-408. http://doi.org/10.1007/s10295-004-0159-0
Velasco, I., Tenreiro, S., Calderon, I. L., & André, B. (2004). Saccharomyces cerevisiae Aqr1 is an internal-membrane transporter involved in excretion of amino acids. Eukaryotic Cell, 3(6), 1492-1503. http://doi.org/10.1128/EC.3.6.1492-1503.2004
Wach, A., Brachat, A., Pohlmann, R., and Philippsen, P. (1994). New heterologous modules for classical or PCR-based gene disruptions in Saccharomyces cerevisiae. Yeast 10, 1793-1808.

1. A yeast strain wherein activity of one or more membrane transporters of the DHA1 family is reduced relative to a wild type strain or to a parental strain from which it is derived.

2. The yeast strain according to claim 1, wherein one or more nucleotide sequences encoding the one or more membrane transporters of the DHA1 family are deleted or inactivated, or wherein expression of said one or more nucleotide sequences is downregulated, preferably inducibly downregulated.

3. The yeast strain according to claim 1, wherein the one or more membrane transporters of the DHA1 family are selected from the group consisting of AQR1, QDR1, QDR2, QDR3, TPO1, TPO2, TPO3, TPO4, FLR1, YHK8, DTR1, HOL1 and/or homologues thereof.

4. The yeast strain according to claim 1, wherein the one or more membrane transporters of the DHA1 family are encoded by a nucleotide sequence comprising, consisting essentially of or consisting of a sequence having at least 60% sequence identity with any of the nucleotide sequences set forth in SEQ ID NOs: 2, 4, 6, 8, 10, 12, 14, 16, 18, 20, 22 and 24; or comprise, consist essentially of or consist of an amino acid sequence having at least 60% identity or similarity with any of the amino acid sequences set forth in SEQ ID NOs: 1, 3, 5, 7, 9, 11, 13, 15, 17, 19, 21 and 23.

5. The yeast strain according to claim 1, having reduced activity of at least two, at least three, at least four, at least five, at least six, or at least seven transporters of the DHA1 family relative to a wild type strain or to a parental strain from which it is derived.

6. The yeast strain according to claim 1, wherein the one or more membrane transporters comprise:
AQR1, QDR2 and QDR3 or homologues thereof;
QDR1, QDR2 and QDR3 or homologues thereof;
AQR1, QDR2, QDR1 and TPO4 or homologues thereof; or
AQR1, QDR3, QDR2, QDR1, DTR1, HOL1 and TPO1, or homologues thereof.

7. The yeast strain according to claim 1, wherein the yeast is a Saccharomyces species, preferably Saccharomyces cerevisiae.

8. A composition comprising, consisting essentially of, or consisting of a yeast strain according to claim 1; and optionally one or more formulation excipients, preferably wherein the composition is a dry yeast composition or an instant yeast composition.

9. A method for the production of yeast cells, comprising culturing a yeast strain as defined in claim 1 in a suitable culture medium.

10. A method for producing a fermentation product, comprising:
culturing a yeast strain as defined in claim 1 on a fermentable substrate; and
optionally, recovering the fermentation product.

11. The method according to claim 10, wherein the fermentation substrate comprises, consists essentially of or consists of an unrefined or low-processed source of sugars.

12. The method according to claim 10, wherein the fermentation product is an alcohol, preferably a lower alkyl alcohol such as ethanol, propanol or butanol, more preferably ethanol.

13. A method for the production of a product of interest, comprising:
a) optionally, transforming a yeast cell according to claim 1 with a vector encoding the product of interest or encoding one or more enzymes capable of producing the product of interest;
b) culturing the yeast cell in a culture medium to produce the product of interest; and
c) optionally, isolating the product of interest from the yeast cell or culture medium.

14. A method for eliminating, reducing or preventing contamination of lactic acid bacteria in yeast cultures, the method comprising culturing a yeast strain as defined in claim 1.

15. The method according to claim 14, wherein the lactic acid bacteria comprise a Lactobacillus species.
